(19) United States
(12) Patent Application Publication
(10) Pub. No.: US 2015/0275275 A1
(43) Pub. Date: Oct. 1, 2015

(54) PROGNOSTIC OF DIET IMPACT ON OBESITY-RELATED CO-MORBIDITIES

(71) Applicants: INSTITUT NATIONAL DE LA RECHERCHE AGRONOMIQUE, Paris (FR); UNIVERSITE PIERRE ET MARIE CURIE (PARIS 6), Paris (FR); INSTITUT DE RECHERCHE POUR LE DEVELOPPEMENT (IRD), MARSEILLE (FR)

(72) Inventors: Stanislav Ehrlich, Orsay (FR); Joel Dore, Vitry Sur Seine (FR); Emmanuelle Le Chatelier, Magny Les Hameaux (FR); Karine Clement, Paris (FR); Jean-Daniel Zucker, Paris (FR)

(21) Appl. No.: 14/435,736

(22) PCT Filed: Oct. 17, 2013
(86) PCT No.: PCT/EP2013/071764

§ 371 Date: Apr. 14, 2015

(30) Foreign Application Priority Data
Oct. 17, 2012 (EP) 12306285.3

(51) Int. Cl. C12Q 1/68

(52) U.S. Cl. CPC C12Q 1/689; C12Q 2600/158; C12Q 2600/16

(57) ABSTRACT

The present invention relates to a method for determining whether an overweight subject has a reduced gut bacterial diversity. The said method comprises detecting the presence or absence in a gut DNA sample of at least one gene of at least one bacterial species of Table 1 or Table 2, respectively.

The present invention relates to methods to determine the risk of obesity-related co-morbidities in overweight subjects based on the gut bacterial microbiome, and methods to increase bacterial richness in individuals in need thereof, particularly in overweight individuals.

Overweight and obesity are defined as abnormal or excessive fat accumulation that presents a risk to health. A crude population measure of obesity is the body mass index (BMI), a person's weight (in kilograms) divided by the square of his or her height (in metres). A person with a BMI equal to or more than 25 is considered overweight. A person with a BMI of 30 or more is generally considered obese.

Overweight and obesity are major risk factors for a number of chronic diseases, or so called obesity-related co-morbidities, including diabetes, cardiovascular diseases and cancer. A substantial literature supports that overweight and obesity are major causes of co-morbidities which can lead to further morbidity and mortality. In a recent review, 18 co-morbidities were shown to be statistically correlated with overweight and obesity: cancer (kidney, colorectal, prostate, ovarian, uterine/endometrial, esophageal, pancreatic, and post-menopausal breast), type II diabetes, cardiovascular disease risk (hypertension, coronary artery disease, congestive heart failure, pulmonary embolism, stroke), gallbladder disease, chronic back pain, osteoarthritis and asthma (Guh D. et al., BMC Public Health.; 9: 88; 2009).

This raises serious concern as regards to public health, both from the point of view of the patients, but also from an economic standpoint. Indeed, the above-mentioned diseases most often require extensive medical treatments, or, in the case of chronic diseases, continuous monitoring.

While it has been long recognized that the most efficient strategy to prevent those diseases is weight loss, many different weight loss strategies can be considered, depending on the patient. While classical low calorie diets have been recommended by practitioners for a long time, drastic approaches such as surgery can be necessary in some cases. There is still on-going discussion as to what kind of weight loss strategy is more efficient to alleviate co-morbidity risks. One of the possibilities that seem to surface from different studies is that specific weight loss strategies may need to be tailored to the overweight or obese individual, depending on several factors such as genetic background and hormonal profile. Yet, probably because of the diversity of cases, no clear markers have been identified that could serve as decision-making help for practitioners.

Recent research however points toward another aspect of the issue at hand, which is that the human microbiota plays a crucial role in both the predispositions of different diseases (Clemente et al., Cell., 148(6):1258-70, 2012).

The human microbiota comprises thousands of bacterial species, among which commensal, beneficial or pathogen bacteria. Humans host microbiota in multiple locations such as skin, lung, vagina, mouth, and gut. Those microbiota are different in their location and in their bacterial composition. The gut microbiota is the largest in its composition. It is generally considered that it comprises thousands of bacterial species, weighs about 1.5 kg and constitutes a rich gene repertoire on its own, also called gut microbiome, 100 times larger than the human nuclear genome.

The gut microbiota has been shown to play a role in the development of metabolic disorders such as obesity, metabolic syndrome, and diabetes. While normbiosis, qualifying the normal state of the microbiota, seems to guaranty homeostasis, disbiosis, which is the distortion from normbiosis, correlates with a long list of diseases.

Recent studies show that the human gut microbiota may be altered in obese relative to lean individuals, even if somewhat inconsistent changes have been reported. An increase in the phylum Firmicutes and a decrease of Bacteroidetes associated with obesity was observed in some, but not all studies, with the inverse also reported. An increase of Actinobacteria in obese was reported as well. Mouse gut microbiota obesity-related alterations are characterized by changes in the Firmicutes to Bacteroidetes ratio, increased in the obese animals. These changes are likely not a mere consequence of obesity, since the obese phenotype can be transmitted by gut microbiota transplantation in mice, indicating that gut microbial populations may have an active role in obesity pathogenesis, and thus probably with the associated co-morbidities.

However, not all of the bacterial species of the gut microbiota have been identified and sequenced, mostly because most of them cannot be cultured. In addition, most bacteria are only present at a low copy number in the gut microbiota, which makes them difficult to detect (Hamady and Knight, Genome Res., 19: 1141-1152, 2009). Therefore, most sequences in the gut bacterial DNA are not yet taxonomically assigned, which restrains the use as biomarkers to taxonomically known species and genes.

There is thus still a need for markers that would allow the determination of the risk for an overweight subject to develop obesity-related co-morbidities, and moreover to differentiate between overweight subjects in order to adapt the weight-loss strategy to each individual.

### FIGURE LEGEND

FIG. 1. Gut microbial composition of LGC (n=18) and HGC (n=27) subjects.

a) Gene count distribution in all individuals (red line) or enterotypes (black lines) at baseline, upon downsizing the data to 7 millions of uniquely matched reads. b) Presence and frequency of 25 tracer genes for 18 bacterial species. Rows correspond to genes and the relative frequency of each gene is indicated by color, increasing from light grey to intense grey; white denotes that a gene has not been detected. Columns correspond to individuals, who are ordered by increasing gene number for the 3 time points of the study. Values on the right side of the figure give the Mann-Whitney probability (q: adjusted by Benjamini-Hochberg method) that a species is differentially abundant among the low and high gene individuals; the abundance of a species in an individual was computed as the mean of frequencies of the tracer genes. F. prau & R. inul stand for F. prausnitzii and R. inulinivorans, respectively. c) AUC values obtained for the best combinations of 1 to 15 species in a ROC analysis of 45 individuals of our cohort (red); Inset: AUC for the best combination of 6 species. AUC French and AUC Danes refer to French (Micro-obese) and the Danish (MetaHIT) cohorts, respectively.

FIG. 2. Differences between LGC (n=18) and HGC (n=27) subjects in bioclinical variables.

White and black bars refer to LGC & HGC groups, respectively; standard errors of the mean are indicated. 0 wk: baseline, 6 wks: end of the energy restriction period, and 12 wks: end of stabilization period. #: p-value<0.1, *: p-value<0.05, **: p-value<0.01 by Maim-Whitney tests. “Disse index” is calculated by combining lipid and insulin values (see supplementary material).

### DESCRIPTION

The inventors have established that the clinical traits linked to obesity-related co-morbidities are significantly correlated to a reduced gut bacterial diversity in overweight subjects. They moreover designed a method to accurately determine whether an overweight subject has a reduced bacterial diversity. It is thereby possible to determine with a high sensitivity whether an overweight subject is at risk to develop obesity-related co-morbidities.

The present invention is directed to a method for determining whether an overweight subject has a reduced gut bacterial diversity is at risk to develop obesity-related co-morbidities. Such a determination is particularly useful to assess if an overweight subject is at risk of developing obesity-related co-morbidities. The inventors have shown that it is possible to discriminate between overweight subjects having reduced gut bacterial diversity and those having normal gut bacterial diversity by simply assessing the presence of a small number of bacterial species in the gut

The inventors have found a set of specific bacterial species, which presence or absence in the bacterial DNA of the faeces of an overweight subject significantly correlates with reduced gut bacterial diversity. These species are not limited to the ones which have already been known from prior art.

By “reduced gut bacterial diversity”, it is herein referred to a gut microbiota in which the number of bacterial species is reduced compared to the average normal gut microbiota.

For example, the comparison between a test microbiota and a normal gut microbiota can be achieved by the genotyping of sequences obtained from the biological samples for example with massively parallel DNA sequencing. In that case, a subject with reduced bacterial diversity can have a microbiome comprising less than 480 000 bacterial gene counts, wherein said counts were obtained by sequencing gut microbial DNA obtained from a sample of 200 mg of faeces with Illumina-based high throughput sequencing, mapping the sequences obtained onto a reference set of bacterial genome (as described in Arumugam et al., Nature., 473(7346):174-80, 2011), removing human contamination, discarding reads mapping at multiple positions, and based on the total amount of remaining matched reads.

According to the invention, a subject has either a reduced gut bacterial diversity, or a normal bacterial diversity. The skilled person would then understand easily that when the method of the invention does not determine that the overweight subject has a reduced gut bacterial diversity, said subject obviously has a normal gut bacterial diversity. By “normal gut bacterial diversity”, it is herein referred to a gut microbiota in which the number of bacterial species is around the number found in the average normal gut microbiota, that is to say between 10% inferior and 10% superior to the number of bacterial species found in the average normal gut microbiota.

By “microbiota”, it is herein referred to microflora and microfauna in an ecosystem such as intestines, mouth, vagina, or lungs. In microbiology, flora (plural: floras or floræ) refers to the collective bacteria and other microorganisms in an ecosystem (e.g., some part of the body of an animal host). The “gut microbiota” consists of all the bacterial species constituting the microbiota present in the gut of an individual.

A bacterial species according to the invention encompasses not only known bacterial species, but also species which have not yet been taxonomically described. Indeed, whether they already have been taxonomically described or not, bacterial species can be characterized by their genome. For example, methods for characterizing bacteria using genetic information have been described in Vandamme et al. (Microbiol. Rev. 1996, 60(2):407).

It will be obvious to the person skilled in the art that the genes of a bacterial species are physically linked as a unit rather than being independently distributed between individuals, i.e. the genome of said bacterial species comprises gene sequences which are always present or absent together among individuals. Bacterial species can therefore be defined by parts of their genome, and sequencing the entire genome of bacterial species is not necessary for proper bacterial species identification.

For instance, a method for the identification of bacterial species in a microbial composition, based on bacterial DNA sequencing and using marker genes as taxonomic references has been described in Liu et al. (BMC genomics, 12(S2):S4, 2011). The person skilled in the art may further refer to Arumugam et al. (Nature, 473(7346):174-80, 2011) or Qin et al. (Nature, 490(7418):55-60, 2012) for detailed methods for the identification of bacterial species based on bacterial DNA sequencing.

According to the present invention a “bacterial species” is a group of bacterial genes from the gut microbiome, which abundance level varies in the same proportion among different individual samples. In other words, a bacterial species according to the invention is a cluster of bacterial gene sequences which abundance levels in samples from distinct subjects are statistically linked rather than being randomly distributed. It will be immediately apparent to the skilled person that such a cluster thus corresponds to a bacterial species.

Genes of the microbiome can be ascribed to a bacterial species by several statistical methods known to the person skilled in the art. Preferably, a statistical method for testing covariance is used for testing whether two genes belong to the same cluster. To this end, the skilled person may use non-parametrical measures of statistical dependence, such as the Spearman's rank correlation coefficient for example. Most preferably, a bacterial species according to the invention is a cluster that comprises gut bacterial genes and that is determined by the method used in Qin et al. (Nature, 490(7418): 55-60, 2012) for identifying metagenomic linkage groups.

By “subject”, it is herein referred to a vertebrate, preferably a mammal, and most preferably a human. By “overweight subject”, it is herein referred to a human being having a body mass index superior to 25 kg/m². The Body mass index is defined as the individual's body mass divided by the square of his or her height. The formulae universally used in medicine produce a unit of measure of kg/m2.

There are several ways to obtain samples of the said subject's gut microbial DNA (Sokol et al., Inflamm. Bowel Dis., 14(6): 858-867, 2008). For example, it is possible to prepare mucosal specimens, or biopsies, obtained by coloscopy. However, coloscopy is an invasive procedure which is ill-defined in terms of collection procedure from study to study. Likewise, it is possible to obtain biopies through surgery. However, even more than coloscopy, surgery is an invasive procedure, which effects on the microbial population are not known. Preferred is the fecal analysis, a procedure which has been reliably been used in the art (Bullock et al., Curr Issues Intest Microbiol.; 5(2): 59-64, 2004; Manichanh et al., Gut, 55: 205-211, 2006; Bakir et al., Int J Syst Evol Microbiol, 56(5): 931-935, 2006; Manichanh et al., Nucl. Acids Res., 36(16): 5180-5188, 2008; Sokol et al., Inflamm. Bowel Dis., 14(6): 858-867, 2008). An example of this procedure is described in the Methods section of the Experimental Examples. Feces contain about 1011 bacterial cells per gram (wet weight) and bacterial cells comprise about 50% of fecal mass. The microbiota of the feces represents primarily the microbiology of the distal large bowel. It is thus possible to isolate and analyze large quantities of microbial DNA from the feces of an individual. By “gut microbial DNA”, it is herein understood the DNA from any of the resident bacterial communities of the human gut. The term “gut microbial DNA” encompasses both coding and non-coding sequences; it is in particular not restricted to complete genes, but also comprises fragments of coding sequences. Fecal analysis is thus a non-invasive procedure, which yields consistent and directly-comparable results from patient to patient.

As explained above, “gut microbiome”, as used herein, refers to the set of bacterial genes from the species constituting the microbiota present in the gut of said subject. The sequences of the microbiome of the invention comprise at least gene sequences from the bacterial gene catalogue published by Qin et al. (Nature, 464: 59-65, 2010). The gene sequences from the catalogue are available from the EMBL (http:///www.bork.embl.de/˜arumugam/Qin_et_al_{—}2010/) and BGI (http://gutmeta.genomics.org.cn) websites.

These species are not limited to the ones which have already been known from prior art. Importantly, these specific bacterial species show a high correlation coefficient with reduced gut bacterial diversity. It is thus possible to determine whether a subject has reduced gut bacterial diversity with a high sensitivity. The sensitivity of a method is the proportion of actual positives which are correctly identified as such, and can be estimated by the area under the ROC (Receiver Operating Characteristic) curve, also called AUC. A receiver operating characteristic (ROC), or simply ROC curve, is a graphical plot which illustrates the performance of a binary classifier system as its discrimination threshold is varied. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. Area Under the Curve (AUC) is a measure of a classifier/test performance across all possible values of the thresholds. The higher the AUC, the better the performance of the test.

The inventors have found that it is not necessary to determine the presence or the absence of every single of the said bacterial species in order to assess the diversity of the gut bacterial population. Rather, said diversity can be evaluated with a high degree of confidence and accuracy by examining a very small subset of bacterial species. As shown in the experimental part, a very small number of species is a good marker of the said diversity. Indeed, even when the presence or absence of only one bacterial species is assessed, the method of the invention enables the detection of reduced bacterial diversity in a subject with an AUC of at least 0.74, and can be up to 0.89, depending of the bacterial species chosen.

In comparison, a random method usually has an AUC of 0.5. Moreover, when inflammatory bowel disease, one of the pathologies associated with reduced bacterial diversity, is assessed by 16S rRNA sequencing of fecal samples, the AUC is of only 0.83 (Papa et al; PLoS One. 2012; 7(6):e39242. 2012).

According to a first embodiment of the invention, the method for determining whether an overweight subject has a reduced gut bacterial diversity comprises a step of detecting from a gut microbial DNA sample obtained from said subject whether at least one gene from at least one bacterial species from Table 1 is absent in said sample.

The bacterial species of the invention are chosen from the list consisting in the bacterial species of Table 1. More precisely, the bacterial species of the invention are chosen from the list consisting in MOHL-1, MOHL-2, MOHL-3, MOHL-4, MOHL-5, MOHL-6, MOHL-7, MOHL-8, MOHL-9, MOHL-10, MOHL-11, MOHL-12, MOHL-13, MOHL-14, MOHL-15, MOHL-16, MOHL-17, MOHL-18.

By “at least one bacterial species”, it is herein meant that the absence of one unique species or of more than one species is assessed. In a preferred embodiment, the method of the invention includes the detection of the absence of 1, 2, 2, 4, or 5 bacterial species. Even more preferably, the said method includes the detection of the absence of more than 5 bacterial species. Most preferably, the said method includes the detection of the absence of 18 bacterial species.

Most intestinal commensals cannot be cultured. Genomic strategies have been developed to overcome this limitation (Hamady and Knight, Genome Res, 19: 1141-1152, 2009). These strategies have allowed the definition of the microbiome as the collection of the genes comprised in the genomes of the microbiota (Turnbaugh et al., Nature, 449: 804-8010, 2007; Hamady and Knight, Genome Res., 19: 1141-1152, 2009). The existence of a small number of species shared by all individuals constituting the human intestinal microbiota phylogenetic core has been demonstrated (Tap et al., Environ Microbiol., 11(10): 2574-2584, 2009). Recently, a metagenomic analysis has led to the identification of an extensive catalogue of 3.3 million non-redundant microbial genes of the human gut, corresponding to 576.7 gigabases of sequence (Qin et al., Nature, 464(7285): 59-65, 2010).

It will be immediately apparent to the person of skills in the art that the presence of a bacterial species can be easily determined by detecting a nucleic acid sequence specific of the said species. The presence of gut bacterial species is usually determined by detecting 16S rRNA gene sequences. However, this method is limited to known bacterial species.

By contrast, in the method of the invention, no prior identification of the bacterial species the said gene belongs to is required. The inventors have determined a minimum set of 25 bacterial gene sequences that are non-redundant sequences for each bacterial species of Table 1, and that can be used as tracer genes.

TABLE 1
bacterial species absent in overweight subjects
with reduced bacterial gut diversity,
and sequences comprised therein.

| Bacterial species | Sequences |
|---|---|
| MOHL-1 | SEQ ID NO. 1 to 25 |
| MOHL-2 | SEQ ID NO. 26 to 50 |
| MOHL-3 | SEQ ID NO. 51 to 75 |
| MOHL-4 | SEQ ID NO. 76 to 100 |
| MOHL-5 | SEQ ID NO. 101 to 125 |
| MOHL-6 | SEQ ID NO. 126 to 150 |
| MOHL-7 | SEQ ID NO. 151 to 175 |
| MOHL-8 | SEQ ID NO. 176 to 200 |
| MOHL-9 | SEQ ID NO. 201 to 225 |
| MOHL-10 | SEQ ID NO. 226 to 250 |
| MOHL-11 | SEQ ID NO. 251 to 275 |
| MOHL-12 | SEQ ID NO. 276 to 300 |
| MOHL-13 | SEQ ID NO. 301 to 325 |
| MOHL-14 | SEQ ID NO. 326 to 350 |
| MOHL-15 | SEQ ID NO. 351 to 375 |
| MOHL-16 | SEQ ID NO. 376 to 400 |
| MOHL-17 | SEQ ID NO. 401 to 425 |
| MOHL-18 | SEQ ID NO. 426 to 450 |

It will be obvious to the person skilled in the art that the number of bacteria from a given bacterial species in a sample directly correlate with the number of copies of at least one gene sequence detected in said sample. It is thereby possible to determine the absence of at least one of the bacterial species from Table 1, simply by detecting the absence of at least one bacterial gene from said species.

The invention therefore enables assessing reduced gut bacterial diversity in an overweight subject, and thereby the risk for said subject to develop obesity-related co-morbidities without the need for complex and tedious statistical analysis. Moreover, because the method of the invention can rely on as little as one bacterial gene as a marker, it may be implemented by any known technique of DNA amplification or sequencing, and is not limited to a specific method or apparatus.

Another embodiment of the invention is a method for determining whether an overweight subject has a reduced gut bacterial diversity, said method comprising:
a) detecting from a gut microbial DNA sample obtained from said subject whether at least one gene from at least one bacterial species from Table 1 is absent in said sample, and
b) determining that the subject has a reduced gut bacterial diversity, if at least one gene from at least one bacterial species from Table 1 is absent in said sample.

In a preferred embodiment, the bacterial genes sequences of the bacterial species according to the invention are chosen in the list consisting of sequence SEQ ID NO. 1 to sequence SEQ ID NO. 450.

Depending on the size of the sample and of the occurrence of the bacterial genes of interest, certain bacterial genes may be difficult to detect in a sample. The skilled person would thus easily conceive that, to increase the confidence of the results, it is advantageous to determine the absence of a bacterial species by detecting the average abundance of several bacterial genes from a bacterial species.

In an embodiment, detecting whether at least one bacterial gene from at least a bacterial species from Table 1 is absent in said sample comprises determining the number of copies of at least 1, 2, 3, 4 or 5 bacterial gene from said bacterial species in the sample. In a preferred embodiment, detecting whether at least one bacterial gene from at least one bacterial species from Table 1 is absent in said sample comprises determining the number of copies of at least 10, 15, 20 or at least 25 bacterial genes from said bacterial species in the sample.

Moreover, among all of the bacterial genes, some bacterial species are more significantly correlated with reduced gut bacterial diversity. For example, as shown in Table 3 of the experimental part, the detection of the presence or absence of one of the bacterial species among MO_HL_{—}5, MO_HL_{—}6, MO_HL_{—}14, MO_HL_{—}9, MO_HL_{—}11, MO_HL_{—}13, MO_HL_{—}3, MO_HL_{—}8, MO_HL_{—}16, MO_HL_{—}4, MO_HL_{—}17, MO_HL_{—}1, or MO_HL_{—}7 enables the detection of reduced bacterial diversity in a subject with an AUC superior to 0.83.

It is thereby possible to increase the sensitivity of the method of the invention, simply by assessing the presence or absence of those specific bacterial species, or of a least one gene from the specific bacterial species they belong to.

In an advantageous embodiment, the method of the invention comprises a step of detecting from a gut microbial DNA sample obtained from said subject whether at least one gene from a bacterial species chosen from the list consisting in MO_HL_{—}5, MO_HL_{—}6, MO_HL_{—}14, MO_HL_{—}9, MO_HL_{—}11, MO_HL_{—}13, MO_HL_{—}3, MO_HL_{—}8, MO_HL_{—}16, MO_HL_{—}4, MO_HL_{—}17, MO_HL_{—}1, or MO_HL_{—}7 from Table 1 is absent in said sample.

In a particularly advantageous embodiment, the method of the invention comprises a step of detecting from a gut microbial DNA sample obtained from said subject whether at least one gene from the bacterial species MOHL-5 from Table 1 is absent in said sample. The person skilled in the art knows that the more distinct bacterial species from Table 1 are present in the bacterial DNA from the feces of the subject, the higher the probability that the subjects has a reduced gut bacterial diversity. It would then be obvious to the skilled person that the sensitivity of the method of the invention can be increased by assessing the absence of bacterial genes from several different bacterial species from Table 1. It is then possible to increase the sensitivity of the method by using bacterial genes from a linear combination of 2, 3, 4, 5 or more different bacterial species. For example, the combinations of 2 bacterial species from Table 1 enable an AUC between around 0.775 and 0.948, the combinations of 3 bacterial species from Table 1 enable an AUC between around 0.813 and 0.993, and the combinations of 4 bacterial species from Table 1 and enable an AUC between around 0.827 and 0.987. However, the inventors have surprisingly discovered that the detection of specific combinations of 2, 3 or 4 bacterial species enables for very high AUC. The more advantageous combinations of 2, 3 and 4 bacterial species are indicated in Table 3, 4 and 5 respectively.

In a preferred embodiment, the method of the invention comprises a step of detecting from a gut microbial DNA sample obtained from said subject whether at least one gene from each of the bacterial species of any of the bacterial species combinations indicated in Table 3, 4 and/or 5 is absent in said sample.

The inventors have additionally selected bacterial species combinations of 2 to 18 bacterial species that enables for particularly important AUC, indicated in Table 6, ranging from around 0.89 to 0.99.

In an advantageous embodiment, the method of the invention comprises a step of detecting from a gut microbial DNA sample obtained from said subject whether:
at least one gene from each of the bacterial species MO_HL_{—}12 and MO_HL_{—}11 from Table 1 are absent in said sample, or;
at least one gene from each of the bacterial species MO_HL_{—}11, MO_HL_{—}7 and MO_HL_{—}12 from Table 1 are absent in said sample, or;
at least one gene from each of the bacterial species MO_HL_{—}11, MO_HL_{—}5, MO_HL_{—}7 and MO_HL_{—}12 from Table 1 are absent in said sample, or;
at least one gene from each of the bacterial species MO_HL_{—}11, MO_HL_{—}8, MO_HL_{—}12, MO_HL_{—}7 and MO_HL_{—}2 from Table 1 are absent in said sample, or;
at least one gene from each of the bacterial species MO_HL_{—}17, MO_HL_{—}5, MO_HL_{—}12, MO_HL_{—}7, MO_HL_{—}11 and MO_HL_{—}8 from Table 1 are absent in said sample, or;
at least one gene from each of the bacterial species MO_HL_{—}12, MO_HL_{—}11, MO_HL_{—}2, MO_HL_{—}5, MO_HL_{—}8, MO_HL_{—}13 and MO_HL_{—}7 from Table 1 are absent in said sample, or;
at least one gene from each of the bacterial species MO_HL_{—}12, MO_HL_{—}2, MO_HL_{—}17, MO_HL_{—}11, MO_HL_{—}13, MO_HL_{—}5, MO_HL_{—}8 and MO_HL_{—}7 from Table 1 are absent in said sample, or;
at least one gene from each of the bacterial species MO_HL_{—}4, MO_HL_{—}11, MO_HL_{—}6, MO_HL_{—}13, MO_HL_{—}2, MO_HL_{—}8, MO_HL_{—}12, MO_HL_{—}7 and MO_HL_{—}17 from Table 1 are absent in said sample, or;
at least one gene from each of the bacterial species MO_HL_{—}8, MO_HL_{—}11, MO_HL_{—}6, MO_HL_{—}4, MO_HL_{—}13, MO_HL_{—}5, MO_HL_{—}2, MO_HL_{—}7, MO_HL_{—}17 and MO_HL_{—}12 from Table 1 are absent in said sample;
at least one gene from each of the bacterial species MO_HL_{—}5, MO_HL_{—}13, MO_HL_{—}4, MO_HL_{—}12, MO_HL_{—}15, MO_HL_{—}17, MO_HL_{—}6, MO_HL_{—}2, MO_HL_{—}8, MO_HL_{—}7 and MO_HL_{—}11 from Table 1 are absent in said sample;
at least one gene from each of the bacterial species MO_HL_{—}11, MO_HL_{—}6, MO_HL_{—}17, MO_HL_{—}4, MO_HL_{—}3, MO_HL_{—}12, MO_HL_{—}5, MO_HL_{—}10, MO_HL_{—}8, MO_HL_{—}7, MO_HL_{—}2 and MO_HL_{—}13 from Table 1 are absent, in said sample;
at least one gene from each of the bacterial species MO_HL_{—}18, MO_HL_{—}15, MO_HL_{—}11, MO_HL_{—}10, MO_HL_{—}5, MO_HL_{—}4, MO_HL_{—}6, MO_HL_{—}8, MO_HL_{—}12, MO_HL_{—}7, MO_HL_{—}2, MO_HL_{—}13 and MO_HL_{—}3 from Table 1 are absent in said sample, or;
at least one gene from each of the bacterial species MO_HL_{—}6, MO_HL_{—}17, MO_HL_{—}3, MO_HL_{—}2, MO_HL_{—}4, MO_HL_{—}18, MO_HL_{—}5, MO_HL_{—}13, MO_HL_{—}10, MO_HL_{—}15, MO_HL_{—}7, MO_HL_{—}1, MO_HL_{—}8 and MO_HL_{—}12 from Table 1 are absent in said sample, or;
at least one gene from each of the bacterial species MO_HL_{—}10, MO_HL_{—}18, MO_HL_{—}2, MO_HL_{—}13, MO_HL_{—}7, MO_HL_{—}1, MO_HL_{—}11, MO_HL_{—}3, MO_HL_{—}4, MO_HL_{—}15, MO_HL_{—}5, MO_HL_{—}6, MO_HL_{—}17, MO_HL_{—}12 and MO_HL_{—}8 from Table 1 are absent, in said sample;
at least one gene from each of the bacterial species MO_HL_{—}7, MO_HL_{—}4, MO_HL_{—}13, MO_HL_{—}1, MO_HL_{—}5, MO_HL_{—}6, MO_HL_{—}11, MO_HL_{—}12, MO_HL_{—}8, MO_HL_{—}3, MO_HL_{—}18, MO_HL_{—}2, MO_HL_{—}10, MO_HL_{—}17, MO_HL_{—}15 and MO_HL_{—}9 from Table 1 are absent in said sample, or;
at least one gene from each of the bacterial species MO_HL_{—}10, MO_HL_{—}8, MO_HL_{—}1, MO_HL_{—}16, MO_HL_{—}18, MO_HL_{—}5, MO_HL_{—}15, MO_HL_{—}2, MO_HL_{—}7, MO_HL_{—}6, MO_HL_{—}13, MO_HL_{—}11, MO_HL_{—}17, MO_HL_{—}3, MO_HL_{—}14, MO_HL_{—}4 and MO_HL_{—}12 from Table 1 are absent, in said sample;
at least one gene from each of the bacterial species MO_HL_{—}15, MO_HL_{—}17, MO_HL_{—}4, MO_HL_{—}14, MO_HL_{—}16, MO_HL_{—}10, MO_HL_{—}7, MO_HL_{—}6, MO_HL_{—}11, MO_HL_{—}9, MO_HL_{—}5, MO_HL_{—}3, MO_HL_{—}8, MO_HL_{—}1, MO_HL_{—}12, MO_HL_{—}18, MO_HL_{—}13 and MO_HL_{—}2 from Table 1 are absent, in said sample.

A bacterial gene is absent from the sample when its number of copies in the sample is inferior to a certain threshold value. Accordingly, a bacterial gene is present in the sample when its number of copies in the sample is inferior to a certain threshold value.

According to the present invention, a “threshold value” is intended to mean a value that permits to discriminate samples in which the number of copies of the bacterial gene of interest is low or high.

In particular, if a number of copies of a bacterial gene of interest is inferior or equal to the threshold value, then the number of copies of this bacterial gene in the sample is considered low, whereas if the number of copies is superior to the threshold value, then the number of copies of this bacterial gene in the sample is considered high. A low copy number means that the bacterial gene is absent from the sample, whereas a high number of copies means that the bacterial gene is present in the sample.

For each gene, and depending on the method used for measuring the number of copies of the bacterial gene, the optimal threshold value may vary. However, it may be easily determined by a skilled person based on the analysis of the microbiome of several individuals in which the number of copies (low or high) is known for this particular bacterial gene, and on the comparison thereof with the number of copies of a control gene. Such a comparison may be facilitated by using the same amount of bacterial DNA for each of the analyzed samples, or by dividing the number of copies of the bacterial gene obtained, by the initial amount of bacterial DNA used in the test. Indeed, it is well known from the skilled person that the total amount of bacteria in the gut of a subject, and consequently in its feces, remains the same even in the case of reduced bacterial diversity. It is also possible to use a reference such as a gut bacterial species whose abundance is known not to vary between individuals with reduced and normal bacterial diversity.

According to the invention, determining the number of copies of at least one bacterial gene in a sample obtained from the subject can be achieved by any technique capable of detecting and quantifying nucleic acids sequences, and include inter alia hybridization with a labelled probe, PCR amplification, sequencing, and all other methods known to the person of skills in the art.

In a first embodiment, determining the number of copies of at least one bacterial gene in a sample obtained from the subject is performed using sequencing. Optionally, DNA is be fragmented, for example by restriction nuclease prior to sequencing. Sequencing is done using any technique known in the state of the art, including sequencing by ligation, pyrosequencing, sequencing-by-synthesis or single-molecule sequencing. Sequencing also includes PCR-Based techniques, such as for example quantitative PCR or emulsion PCR.

Sequencing is performed on the entire DNA contained in the biological sample, or on portions of the DNA contained in the biological sample. It will be immediately clear to the skilled person that the said sample contains at least a mixture of bacterial DNA and of human DNA from the host subject. However, though the overall bacterial DNA is likely to represent the major fraction of the total DNA present in the sample, each bacterial species may only represent a small fraction of the total DNA present in the sample.

To overcome this difficulty, the skilled person can use a method that allows the quantitative genotyping of sequences obtained from the biological sample with high precision. In one embodiment of this approach, the precision is achieved by analysis of a large number (for example, millions or billions) of polynucleotides. Furthermore, the precision can be enhanced by the use of massively parallel DNA sequencing, such as, but not limited to that performed by the Illumina Genome Analyzer platform (Bentley et al. Nature; 456: 53-59, 2008), the Roche 454 platform (Margulies et al. Nature; 437: 376-380, 2005), the ABI SOLiD platform (McKernan et al., Genome Res; 19: 1527-1541, 2009), the Helicos single molecule sequencing platform (Harris et al. Science; 320: 106-109, 2008), real-time sequencing using single polymerase molecules (Science; 323: 133-138, 2009), Ion Torrent sequencing (WO 2010/008480; Rothberg et al., Nature, 475: 348-352, 2011) and nanopore sequencing (Clarke J et al. Nat Nanotechnol.; 4: 265-270, 2009).

When the skilled person relies on sequencing methods to detect the presence or absence of certain bacterial genes, the information collected from sequencing is used to determine the number of copies of nucleic acid sequences of interest via bioinformatics procedures. For example, in an embodiment, the nucleic acid sequences of said bacterial species in the gut bacterial DNA sample are identified in the global sequencing data by comparison with the nucleic acid sequences SEQ ID NO. 1 to SEQ ID NO. 450. This comparison is advantageously based on the level of sequence identity with the sequences SEQ ID NO. 1 to SEQ ID NO. 450.

Thus, a nucleic acid sequence displaying at least 90%, at least 95%, at least 96%, at least 97%, at least 98%, at least 99%, or 100% identity with at least one of the nucleic acid sequences SEQ ID NO. 1 to SEQ ID NO. 450 is identified as a sequence comprised in one of the bacterial species of the invention.

Thus, in a preferred embodiment, detecting whether at least one bacterial species from Table 1 is absent in said sample comprises determining the number of nucleic acid sequences in the gut bacterial DNA sample having at least 90%, at least 95%, at least 96%, at least 97%, at least 98%, at least 99%, or 100% identity with at least one of the nucleic acid sequences SEQ ID NO. 1 to SEQ ID NO. 450.

The term “sequence identity” herein refers to the identity between two nucleic acids sequences. Identity between sequences can be determined by comparing a position in each of the sequences which may be aligned for the purposes of comparison. When a position in the compared sequences is occupied by the same base, then the sequences are identical at that position. A degree of sequence identity between nucleic acid sequences is a function of the number of identical nucleotides at positions shared by these sequences.

To determine the percent identity of two amino acids sequences, the sequences are aligned for optimal comparison. For example, gaps can be introduced in the sequence of a first nucleic acid sequence for optimal alignment with the second nucleic acid sequence. The nucleotides at corresponding nucleotide positions are then compared. When a position in the first sequence is occupied by the same nucleotide as the corresponding position in the second sequence, the molecules are identical at that position. The percent identity between the two sequences is a function of the number of identical positions shared by the sequences. Hence % identity=number of identical positions/total number of overlapping positions×100.

In this comparison the sequences can be the same length or can be different in length. Optimal alignment of sequences for determining a comparison window may be conducted by the local homology algorithm of Smith and Waterman (J. Theor. Biol., 91(2): 370-380, 1981), by the homology alignment algorithm of Needleman and Wunsch (J. Mol. Biol, 48(3): 443-453, 1972), by the search for similarity via the method of Pearson and Lipman (Proc. Natl. Acad. Sci. U.S.A., 85(5): 2444-2448, 1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection. The best alignment (i.e. resulting in the highest percentage of identity over the comparison window) generated by the various methods is selected.

The term “sequence identity” thus means that two polynucleotide sequences are identical (i.e. on a nucleotide by nucleotide basis) over the window of comparison. The term “percentage of sequence identity” is calculated by comparing two optimally aligned sequences over the window of comparison, determining the number of positions at which the identical nucleic acid base (e.g. A, T, C, G, U, or I) occurs in both sequences to yield the number of matched positions, dividing the number of matched positions by the total number of positions in the window of comparison (i.e. the window size) and multiplying the result by 100 to yield the percentage of sequence identity. The same process can be applied to polypeptide sequences. The percentage of sequence identity of a nucleic acid sequence or an amino acid sequence can also be calculated using BLAST software (Version 2.06 of September 1998) with the default or user defined parameter.

In another preferred embodiment, PCR-based techniques are used to determine the number of copies of at least one bacterial gene. Preferably, the PCR technique used quantitatively measures starting amounts of DNA, cDNA, or RNA. Examples of PCR-based techniques according to the invention include techniques such as, but not limited to, quantitative PCR (Q-PCR), reverse-transcriptase polymerase chain reaction (RT-PCR), quantitative reverse-transcriptase PCR (QRT-PCR), rolling circle amplification (RCA) or digital PCR. These techniques are well known and easily available technologies for those skilled in the art and do not need a precise description. In a preferred embodiment, the determination of the copy number of the bacterial genes of the invention is performed by quantitative PCR.

Amplification primers specific for the genes to be tested are thus also very useful for performing the methods according to the invention. The present invention thus also encompasses primers for amplifying at least one gene selected from the genes of sequence SEQ ID NO. 1-450.

In another preferred embodiment, the presence or absence of the bacterial genes according to the invention is detected by the use of a nucleic microarray.

According to the invention, a “nucleic microarray” consists of different nucleic acid probes that are attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes can be nucleic acids such as cDNAs (“cDNA microarray”) or oligonucleotides (“oligonucleotide microarray”), and the oligonucleotides may be about 25 to about 60 base pairs or less in length.

To determine the copy number of a target nucleic sample, said sample is labelled, contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the man skilled in the art.

In a specific embodiment, the nucleic microarray is an oligonucleotide microarray comprising at least one oligonucleotide specific for at least one gene having a sequence selected from SEQ ID NOs 1-450. Preferably, the said microarray comprises at least 18 oligonucleotides, each oligonucleotide being specific for one gene of a distinct cluster of the invention. More preferably, the microarray of the invention consists of 450 oligonucleotides specific for each of the genes of sequences SEQ ID NOs. 1-450.

In another embodiment, the nucleic microarray is an oligonucleotide microarray comprising at least one oligonucleotide specific for at least one gene of each of the bacterial species MO_HL_{—}12 and MO_HL_{—}11. Preferably, the nucleic microarray is an oligonucleotide microarray comprising or consisting in oligonucleotides specific for at least 2, 3, 4, 5, 10, 20, or 25 genes of each of the bacterial species MO_HL_{—}12 and MO_HL_{—}11.

In another embodiment, the nucleic microarray is an oligonucleotide microarray comprising at least one oligonucleotide specific for at least one gene of each of the bacterial species MO_HL_{—}11, MO_HL_{—}7 and MO_HL_{—}12. Preferably, the nucleic microarray is an oligonucleotide microarray comprising or consisting in oligonucleotides specific for at least 2, 3, 4, 5, 10, 20, or 25 genes of each of the bacterial species MO_HL_{—}11, MO_HL_{—}7 and MO_HL_{—}12.

In another embodiment, the nucleic microarray is an oligonucleotide microarray comprising at least one oligonucleotide specific for at least one gene of each of the bacterial species MO_HL_{—}11, MO_HL_{—}5, MO_HL_{—}7 and MO_HL_{—}12. Preferably, the nucleic microarray is an oligonucleotide microarray comprising or consisting in oligonucleotides specific for at least 2, 3, 4, 5, 10, 20, or 25 genes of each of the bacterial species MO_HL_{—}11, MO_HL_{—}5, MO_HL_{—}7 and MO_HL_{—}12.

In another embodiment, the nucleic microarray is an oligonucleotide microarray comprising at least one oligonucleotide specific for at least one gene of each of the bacterial species MO_HL_{—}11, MO_HL_{—}8, MO_HL_{—}12, MO_HL_{—}7 and MO_HL_{—}2. Preferably, the nucleic microarray is an oligonucleotide microarray comprising or consisting in oligonucleotides specific for at least 2, 3, 4, 5, 10, 20, or 25 genes of each of the bacterial species MO_HL_{—}11, MO_HL_{—}8, MO_HL_{—}12, MO_HL_{—}7 and MO_HL_{—}2

In another embodiment, the nucleic microarray is an oligonucleotide microarray comprising at least one oligonucleotide specific for at least one gene of each of the bacterial species MO_HL_{—}17, MO_HL_{—}5, MO_HL_{—}12, MO_HL_{—}7, MO_HL_{—}11 and MO_HL_{—}8. Preferably, the nucleic microarray is an oligonucleotide microarray comprising or consisting in oligonucleotides specific for at least 2, 3, 4, 5, 10, 20, or 25 genes of each of the bacterial species MO_HL_{—}17, MO_HL_{—}5, MO_HL_{—}12, MO_HL_{—}7, MO_HL_{—}11 and MO_HL_{—}8.

In another embodiment, the nucleic microarray is an oligonucleotide microarray comprising at least one oligonucleotide specific for at least one gene of each of the bacterial species MO_HL_{—}12, MO_HL_{—}11, MO_HL_{—}2, MO_HL_{—}5, MO_HL_{—}8, MO_HL_{—}13 and MO_HL_{—}7. Preferably, the nucleic microarray is an oligonucleotide microarray comprising or consisting in oligonucleotides specific for at least 2, 3, 4, 5, 10, 20, or 25 genes of each of the bacterial species MO_HL_{—}12, MO_HL_{—}11, MO_HL_{—}2, MO_HL_{—}5, MO_HL_{—}8, MO_HL_{—}13 and MO_HL_{—}7.

In another embodiment, the nucleic microarray is an oligonucleotide microarray comprising at least one oligonucleotide specific for at least one gene of each of the bacterial species MO_HL_{—}12, MO_HL_{—}2, MO_HL_{—}17, MO_HL_{—}11, MO_HL_{—}13, MO_HL_{—}5, MO_HL_{—}8 and MO_HL_{—}7. Preferably, the nucleic microarray is an oligonucleotide microarray comprising or consisting in oligonucleotides specific for at least 2, 3, 4, 5, 10, 20, or 25 genes of each of the bacterial species MO_HL_{—}12, MO_HL_{—}2, MO_HL_{—}17, MO_HL_{—}11, MO_HL_{—}13, MO_HL_{—}5, MO_HL_{—}8 and MO_HL_{—}7.

In another embodiment, the nucleic microarray is an oligonucleotide microarray comprising at least one oligonucleotide specific for at least one gene of each of the bacterial species MO_HL_{—}4, MO_HL_{—}11, MO_HL_{—}6, MO_HL_{—}13, MO_HL_{—}2, MO_HL_{—}8, MO_HL_{—}12, MO_HL_{—}7 and MO_HL_{—}17. Preferably, the nucleic microarray is an oligonucleotide microarray comprising or consisting in oligonucleotides specific for at least 2, 3, 4, 5, 10, 20, or 25 genes of each of the bacterial species MO_HL_{—}4, MO_HL_{—}11, MO_HL_{—}6, MO_HL_{—}13, MO_HL_{—}2, MO_HL_{—}8, MO_HL_{—}12, MO_HL_{—}7 and MO_HL_{—}17.

In another embodiment, the nucleic microarray is an oligonucleotide microarray comprising at least one oligonucleotide specific for at least one gene of each of the bacterial species MO_HL_{—}8, MO_HL_{—}11, MO_HL_{—}6, MO_HL_{—}4, MO_HL_{—}13, MO_HL_{—}5, MO_HL_{—}2, MO_HL_{—}7, MO_HL_{—}17 and MO_HL_{—}12. Preferably, the nucleic microarray is an oligonucleotide microarray comprising or consisting in oligonucleotides specific for at least 2, 3, 4, 5, 10, 20, or 25 genes of each of the bacterial species MO_HL_{—}8, MO_HL_{—}11, MO_HL_{—}6, MO_HL_{—}4, MO_HL_{—}13, MO_HL_{—}5, MO_HL_{—}2, MO_HL_{—}7, MO_HL_{—}17 and MO_HL_{—}12.

In another embodiment, the nucleic microarray is an oligonucleotide microarray comprising at least one oligonucleotide specific for at least one gene of each of the bacterial species MO_HL_{—}5, MO_HL_{—}13, MO_HL_{—}4, MO_HL_{—}12, MO_HL_{—}15, MO_HL_{—}17, MO_HL_{—}6, MO_HL_{—}2, MO_HL_{—}8, MO_HL_{—}7 and MO_HL_{—}11. Preferably, the nucleic microarray is an oligonucleotide microarray comprising or consisting in oligonucleotides specific for at least 2, 3, 4, 5, 10, 20, or 25 genes of each of the bacterial species MO_HL_{—}5, MO_HL_{—}13, MO_HL_{—}4, MO_HL_{—}12, MO_HL_{—}15, MO_HL_{—}17, MO_HL_{—}6, MO_HL_{—}2, MO_HL_{—}8, MO_HL_{—}7 and MO_HL_{—}11.

In another embodiment, the nucleic microarray is an oligonucleotide microarray comprising at least one oligonucleotide specific for at least one gene of each of the bacterial species MO_HL_{—}11, MO_HL_{—}6, MO_HL_{—}17, MO_HL_{—}4, MO_HL_{—}3, MO_HL_{—}12, MO_HL_{—}5, MO_HL_{—}10, MO_HL_{—}8, MO_HL_{—}7, MO_HL_{—}2 and MO_HL_{—}13. Preferably, the nucleic microarray is an oligonucleotide microarray comprising or consisting in oligonucleotides specific for at least 2, 3, 4, 5, 10, 20, or 25 genes of each of the bacterial species MO_HL_{—}11, MO_HL_{—}6, MO_HL_{—}17, MO_HL_{—}4, MO_HL_{—}3, MO_HL_{—}12, MO_HL_{—}5, MO_HL_{—}10, MO_HL_{—}8, MO_HL_{—}7, MO_HL_{—}2 and MO_HL_{—}13.

In another embodiment, the nucleic microarray is an oligonucleotide microarray comprising at least one oligonucleotide specific for at least one gene of each of the bacterial species MO_HL_{—}18, MO_HL_{—}15, MO_HL_{—}11, MO_HL_{—}10, MO_HL_{—}5, MO_HL_{—}4, MO_HL_{—}6, MO_HL_{—}8, MO_HL_{—}12, MO_HL_{—}7, MO_HL_{—}2, MO_HL_{—}13 and MO_HL_{—}3 Preferably, the nucleic microarray is an oligonucleotide microarray comprising or consisting in oligonucleotides specific for at least 2, 3, 4, 5, 10, 20, or 25 genes of each of the bacterial species MO_HL_{—}18, MO_HL_{—}15, MO_HL_{—}11, MO_HL_{—}10, MO_HL_{—}5, MO_HL_{—}4, MO_HL_{—}6, MO_HL_{—}8, MO_HL_{—}12, MO_HL_{—}7, MO_HL_{—}2, MO_HL_{—}13 and MO_HL_{—}3.

In another embodiment, the nucleic microarray is an oligonucleotide microarray comprising at least one oligonucleotide specific for at least one gene of each of the bacterial species MO_HL_{—}6, MO_HL_{—}17, MO_HL_{—}3, MO_HL_{—}2, MO_HL_{—}4, MO_HL_{—}18, MO_HL_{—}5, MO_HL_{—}13, MO_HL_{—}10, MO_HL_{—}15, MO_HL_{—}7, MO_HL_{—}1, MO_HL_{—}8 and MO_HL_{—}12. Preferably, the nucleic microarray is an oligonucleotide microarray comprising or consisting in oligonucleotides specific for at least 2, 3, 4, 5, 10, 20, or 25 genes of each of the bacterial species MO_HL_{—}6, MO_HL_{—}17, MO_HL_{—}3, MO_HL_{—}2, MO_HL_{—}4, MO_HL_{—}18, MO_HL_{—}5, MO_HL_{—}13, MO_HL_{—}10, MO_HL_{—}15, MO_HL_{—}7, MO_HL_{—}1, MO_HL_{—}8 and MO_HL_{—}12.

In another embodiment, the nucleic microarray is an oligonucleotide microarray comprising at least one oligonucleotide specific for at least one gene of each of the bacterial species MO_HL_{—}10, MO_HL_{—}18, MO_HL_{—}2, MO_HL_{—}13, MO_HL_{—}7, MO_HL_{—}1, MO_HL_{—}11, MO_HL_{—}3, MO_HL_{—}4, MO_HL_{—}15, MO_HL_{—}5, MO_HL_{—}6, MO_HL_{—}17, MO_HL_{—}12 and MO_HL_{—}8 Preferably, the nucleic microarray is an oligonucleotide microarray comprising or consisting in oligonucleotides specific for at least 2, 3, 4, 5, 10, 20, or 25 genes of each of the bacterial species MO_HL_{—}10, MO_HL_{—}18, MO_HL_{—}2, MO_HL_{—}13, MO_HL_{—}7, MO_HL_{—}1, MO_HL_{—}11, MO_HL_{—}3, MO_HL_{—}4, MO_HL_{—}15, MO_HL_{—}5, MO_HL_{—}6, MO_HL_{—}17, MO_HL_{—}12 and MO_HL_{—}8.

In another embodiment, the nucleic microarray is an oligonucleotide microarray comprising at least one oligonucleotide specific for at least one gene of each of the bacterial species MO_HL_{—}7, MO_HL_{—}4, MO_HL_{—}13, MO_HL_{—}1, MO_HL_{—}5, MO_HL_{—}6, MO_HL_{—}11, MO_HL_{—}12, MO_HL_{—}8, MO_HL_{—}3, MO_HL_{—}18, MO_HL_{—}2, MO_HL_{—}10, MO_HL_{—}17, MO_HL_{—}15 and MO_HL_{—}9. Preferably, the nucleic microarray is an oligonucleotide microarray comprising or consisting in oligonucleotides specific for at least 2, 3, 4, 5, 10, 20, or 25 genes of each of the bacterial species MO_HL_{—}7, MO_HL_{—}4, MO_HL_{—}13, MO_HL_{—}1, MO_HL_{—}5, MO_HL_{—}6, MO_HL_{—}11, MO_HL_{—}12, MO_HL_{—}8, MO_HL_{—}3, MO_HL_{—}18, MO_HL_{—}2, MO_HL_{—}10, MO_HL_{—}17, MO_HL_{—}15 and MO_HL_{—}9.

In another embodiment, the nucleic microarray is an oligonucleotide microarray comprising at least one oligonucleotide specific for at least one gene of each of the bacterial species MO_HL_{—}10, MO_HL_{—}8, MO_HL_{—}1, MO_HL_{—}16, MO_HL_{—}18, MO_HL_{—}5, MO_HL_{—}15, MO_HL_{—}2, MO_HL_{—}7, MO_HL_{—}6, MO_HL_{—}13, MO_HL_{—}11, MO_HL_{—}17, MO_HL_{—}3, MO_HL_{—}14, MO_HL_{—}4 and MO_HL_{—}12. Preferably, the nucleic microarray is an oligonucleotide microarray comprising or consisting in oligonucleotides specific for at least 2, 3, 4, 5, 10, 20, or 25 genes of each of the bacterial species MO_HL_{—}10, MO_HL_{—}8, MO_HL_{—}1, MO_HL_{—}16, MO_HL_{—}18, MO_HL_{—}5, MO_HL_{—}15, MO_HL_{—}2, MO_HL_{—}7, MO_HL_{—}6, MO_HL_{—}13, MO_HL_{—}11, MO_HL_{—}17, MO_HL_{—}3, MO_HL_{—}14, MO_HL_{—}4 and MO_HL_{—}12.

In another embodiment, the nucleic microarray is an oligonucleotide microarray comprising at least one oligonucleotide specific for at least one gene of each of the bacterial species MO_HL_{—}15, MO_HL_{—}17, MO_HL_{—}4, MO_HL_{—}14, MO_HL_{—}16, MO_HL_{—}10, MO_HL_{—}7, MO_HL_{—}6, MO_HL_{—}11, MO_HL_{—}9, MO_HL_{—}5, MO_HL_{—}3, MO_HL_{—}8, MO_HL_{—}1, MO_HL_{—}12, MO_HL_{—}18, MO_HL_{—}13 and MO_HL_{—}2. Preferably, the nucleic microarray is an oligonucleotide microarray comprising or consisting in oligonucleotides specific for at least 2, 3, 4, 5, 10, 20, or 25 genes of each of the bacterial species MO_HL_{—}15, MO_HL_{—}17, MO_HL_{—}4, MO_HL_{—}14, MO_HL_{—}16, MO_HL_{—}10, MO_HL_{—}7, MO_HL_{—}6, MO_HL_{—}11, MO_HL_{—}9, MO_HL_{—}5, MO_HL_{—}3, MO_HL_{—}8, MO_HL_{—}1, MO_HL_{—}12, MO_HL_{—}18, MO_HL_{—}13 and MO_HL_{—}2.

Said microarray may further comprise at least one oligonucleotide for detecting at least one gene of at least one control bacterial species. A convenient bacterial species may be e.g. a bacterial species whose abundance does not vary between individuals with a reduced bacterial diversity and individuals with normal bacterial diversity. Preferably, the oligonucleotides are about 50 bases in length.

SuiTable microarray oligonucleotides specific for any gene of SEQ ID NOs. 1-450 may be designed, based on the genomic sequence of each gene, using any method of microarray oligonucleotide design known in the art. In particular, any available software developed for the design of microarray oligonucleotides may be used, such as, for instance, the OligoArray software (available at http://berry.engin.umich edu/oligoarray/), the GoArrays software (available at http://www.isima.fr/bioinfo/goarrays/), the Array Designer software (available at http://www.premierbiosoft.com/dnamicroarray/index.html), the Primer3 software (available at http://frodo.wi.mit.edu/primer3/primer3_code.html), or the Promide software (available at http://oligos.molgen.mpg.de/).

The invention further concerns a kit for the in vitro determination of the reduced gut bacterial diversity phenotype, comprising at least one reagent for the determination of the copy number of at least one gene having a sequence selected from SEQ ID NOs. 1-450. By “a reagent for the determination of the copy number of at least one gene”, it is meant a reagent which specifically allows for the determination of the copy number of the said gene, i.e. a reagent specifically intended for the specific determination of the copy number of at least one gene having a sequence selected from SEQ ID NOs. 1-450. This definition excludes generic reagents useful for the determination of the expression level of any gene, such as Taq polymerase or an amplification buffer, although such reagents may also be included in a kit according to the invention. Such a reagent for the determination of the copy number of at least one gene can be for example a dedicated microarray as described above or amplification primers specific for at least one gene having a sequence selected from SEQ ID NOs. 1-450. The present invention thus also relates to a kit for the in vitro determination of the reduced gut bacterial diversity phenotype, said kit comprising a dedicated microarray as described above or amplification primers specific for at least one gene having a sequence selected from SEQ ID NOs. 1-450. Here also, when the kit comprises amplification primers, while said kit may comprise amplification primers specific for other genes, said kit preferably comprises at most 100, at most 75, 50, at most 40, at most 30, preferably at most 25, at most 20, at most 15, more preferably at most 10, at most 8, at most 6, even more preferably at most 5, at most 4, at most 3 or even 2 or one or even zero couples of amplification primers specific for other genes than the genes of sequences SEQ ID NOs 1-450. For example, said kit may comprise at least a couple of amplification primers for at least one gene in addition to the primers for at least one gene having a sequence selected from SEQ ID NOs. 1-450.

Such a kit for the in vitro determination of the reduced gut bacterial diversity phenotype may further comprise instructions for detection of the presence or absence of a responsive phenotype.

As the inventors have shown, overweight subject with a reduced gut bacterial diversity present a higher risk of obesity-associated co-morbidities. Determining that an overweight subject has a reduced gut bacterial diversity is therefore extremely useful in the determination of such risk, particularly as it is realized entirely in vitro.

Another object of the invention is therefore a method for determining if an overweight subject is at risk to develop obesity related co-morbidities, said method comprising the steps of:
a) determining that said subject has a reduced gut bacterial diversity with a method according to the invention;
b) if said subject has a reduced gut bacterial diversity, determining that said overweight subject is at is at risk to develop obesity related co-morbidities.

Obesity related co-morbidities are well known of the skilled person. They have been thoroughly studied in scientific literature, and a comprehensive list of them may be found in Guh D. et al, BMC Public Health., 9:88, 2009. The most significant of them are type II diabetes, all cancers except esophageal, pancreatic and prostate cancer, all cardiovascular diseases (except congestive heart failure), asthma, gallbladder disease, osteoarthritis and chronic back pain.

Thus, in an embodiment, the method of the invention is for determining if an overweight subject is at risk to develop type II diabetes, all cancers except esophageal, pancreatic and prostate cancer, all cardiovascular diseases (except congestive heart failure), asthma, gallbladder disease, osteoarthritis and chronic back pain.

Moreover, the inventors have found that the gut bacterial microbiome of overweigth subject with a reduced gut bacterial diversity can be enriched by modifications of their diet, that is to say by implementing a specific diet. Indeed, they discovered that gut bacterial diversity, can be increased with a low calorie, protein-rich diet. The inventors showed that the gut bacterial diversity increase due to the implementation of said diet is more significant in overweight subjects with a reduced bacterial diversity. Those subjects are therefore particularly prone to benefit from dietary intervention.

Another object of the invention is therefore a method for determining if an overweight subject is in need of a diet, said method comprising the steps of:
a) determining that said subject has a reduced gut bacterial diversity with a method of the invention;
b) if said subject has a reduced gut bacterial diversity, determining that said subject is in need of a diet.

In a preferred embodiment, the diet of the invention is a low calorie protein rich diet. In a more preferred embodiment, the diet of the invention comprises diets of between 1,200 and 1,500 Kcal per day, wherein the proteins represent 35% of the total amount of calories, the lipids represent 25% of the total amount of calories, the carbohydrates represent 44% of the total amount of calories. Advantageously, if the overweight subject is a woman, the diet comprises 1,200 Kcal per day and if the overweight subject is a man, the diet comprises 1,500 Kcal per day.

Importantly, this enrichment in gut bacterial species is correlated with an alleviation of the clinical traits linked to obesity-related co-morbidities, and thus the risk to develop obesity-related co-morbidities, except for low grade inflammation.

Another object of the invention is a method for alleviating the risks to develop obesity-related co-morbidities, said method comprising the steps of:
a) determining that said subject has a reduced gut bacterial diversity with a method of the invention;
b) if said subject has a reduced gut bacterial diversity, determining that said subject is in need of a diet.

It will be immediately apparent to the person skilled in the art that overweight subjects with a reduced gut bacterial diversity that also present low grade inflammation may only partially benefit from implementing a diet. It is however particularly important to treat low-grade inflammation as soon as possible and therefore to be able to distinguish between overweight subjects for which low grade inflammation can efficiently be alleviated by dietary intervention only, and those whose low-grade inflammation requires additional treatments. The method of the invention allows for an early detection of those subjects that will require low-grade inflammation treatments in addition to the implementation of the diet.

Another object of the invention is therefore a method for determining if an overweight subject is in need of a low-grade inflammation treatment, preferably in addition to the implementation of a diet, comprising the steps of:
a) determining that said subject has a reduced gut bacterial diversity with a method of the invention;
b) if said subject has a reduced gut bacterial diversity, determining that said subject is in need of a low-grade inflammation treatment, preferably in addition to the implementation of a diet.

The additional low-grade inflammation treatments can be any conventional anti-inflammation treatment such as the lipid lowering 3-hydroxy-3-methylglutaryl coenzyme A (HMG-Co A) reductase inhibitors (statins) and also the insulin sensitizing peroxisome proliferator activated receptor γ activators (thiazolidinediones).

The evolution of the bacterial diversity with implementation of those modifications of the diet can easily be monitored with the method of the invention.

Another object of the invention is therefore a method for monitoring the efficiency of a diet in increasing gut bacterial microbiome richness in an overweight subject in need thereof, said method comprising the steps of:
a) determining that said subject has a reduced gut bacterial diversity with a method of the invention;
b) implementing said diet;
c) determining from a second gut microbial DNA sample obtained from said subject if said subject has a reduced gut bacterial diversity with a method of the invention;
d) if said subject has a reduced gut bacterial diversity, determining that the diet is efficient in increasing gut bacterial microbiome richness in said subject

The practice of the invention employs, unless other otherwise indicated, conventional techniques or protein chemistry, molecular virology, microbiology, recombinant DNA technology, and pharmacology, which are within the skill of the art. Such techniques are explained fully in the literature. (See Ausubel et al., Current Protocols in Molecular Biology, Eds., John Wiley & Sons, Inc. New York, 1995; Remington's Pharmaceutical Sciences, 17th ed., Mack Publishing Co., Easton, Pa., 1985; and Sambrook et al., Molecular cloning: A laboratory manual 2nd edition, Cold Spring Harbor Laboratory Press—Cold Spring Harbor, N.Y., USA, 1989). The nomenclatures used in connection with, and the laboratory procedures and techniques of, molecular and cellular biology, protein biochemistry, enzymology and medicinal and pharmaceutical chemistry described herein are those well-known and commonly used in the art.

Having generally described this invention, a further understanding of characteristics and advantages of the invention can be obtained by reference to certain specific examples and figures which are provided herein for purposes of illustration only and are not intended to be limiting unless otherwise specified.

### Examples

### Clinical Investigation

Obese (n=38) and overweight (n=11) subjects, 8 men and 41 women, were recruited for a 12-week controlled dietary intervention at the Center of Research in Human Nutrition, Pitié-Salpêtrière Hospital, Paris, France. The subjects included in the study had no chronic pathology except body weight excess. Their body weight was stable within 3 months prior to the study. No antibiotics or drugs were taken within 2 months before or during the course of the study. The Ethical Committee of Pitié-Salpêtrière Hospital approved the clinical study and subjects gave written informed consent. In the first 6-week phase, subjects consumed an energy restricted high protein diet (1,200 Kcal/d for women and 1,500 for men: 35% proteins, 25% lipids, 44% carbohydrates) with low glycaemic index carbohydrates and enrichment with soluble fibers. This phase was followed by a second 6-week body weight stabilization period with 20% increase in total energy intake, above their resting energy metabolic rate (indirect calorimetry, Deltatrac, Datex, France). At 0, 6 and 12 weeks, blood and fecal samples were collected and anthropometric measurements were performed. Subjects filled a 7-day dietary record and were interviewed by a registered dietitian. On the visit day, the dietitian checked the information and clarified any ambiguities regarding detail of food consumed. All records were analyzed by the registered dietitian using the computer software program PROFILE DOSSIER V3 (Audit Conseil en Informatique Médicale, Bourges, France), which has a dietary database initially made up of 400 food items representative of the French diet as described previously². A nutrient analysis was generated for each subject. Body composition was determined by dual-energy X-ray absorptiometry (DEXA). Blood samples were obtained after 12 hr of fasting to measure total cholesterol, high-density lipoprotein (HDL) cholesterol, triglycerides, insulin, glucose, and inflammatory markers (highly sensitive C-reactive protein (hs-CRP) and interleukin 6 (IL-6)) as previously described³. Insulin resistance was estimated using homeostasis model assessment of insulin resistance (HOMA-IR) and “Disse” index^{4,5}. Subcutaneous abdominal adipose tissue samples were obtained at all time points by needle biopsy from the periumbilical area under local anesthesia (1% xylocaine) for measuring the adipocytes diameter⁶ and for immunohistochemical studies (HAM56+ stained macrophages in adipose tissue). Paired Wilcoxon tests were performed to analyze changes in these variables between various time points (p-value<0.05). P-values were adjusted for multiple testing using the Benjamini-Hochberg procedure⁷.

### Metagenomic Sequencing

Intestinal bacterial gene content of 49 obese and overweight individuals at 3 time-points (baseline, week 6 and week 12) was determined by high throughput ABI SOLiD sequencing technology of total fecal DNA. An average of 76.5±36.5 (mean±sd) million 35 base-long single reads were determined for each sample (a total of 393 Gb of sequence). By using corona_lite (v4.0r2.0), an average of 24.8±14.3 million reads per individuals were mapped on the reference catalog of 3.3 million genes⁸ with a maximum of 3 mismatches. Reads mapping at multiple positions were discarded and an average of 14.2±8.1 million uniquely mapped reads per individuals were retained for estimating the abundance of each reference gene by using METEOR⁹. Abundance of each gene in an individual was normalized with METEOR by dividing the number of reads that uniquely mapped to the gene of interest by the gene length. After that, normalized gene abundances were transformed in frequencies by dividing them with the total number of uniquely mapped reads for a given sample. The resulting set of gene frequencies, termed as microbial gene profile of an individual, was used for further analyses.

### Differentially Abundant Species Between LGC and HGC

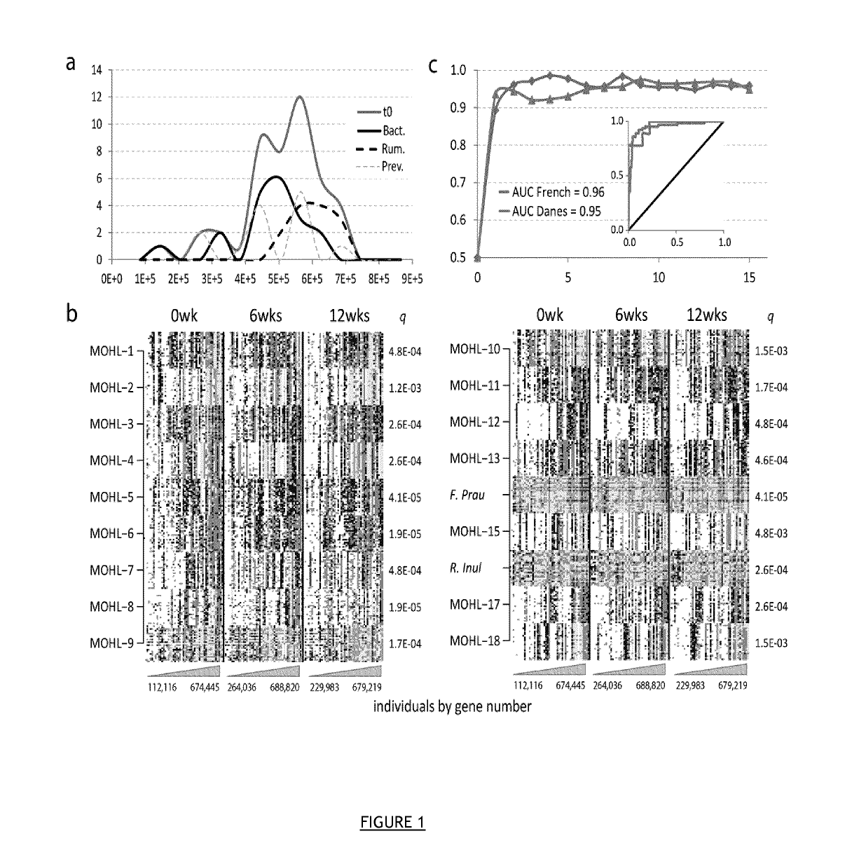

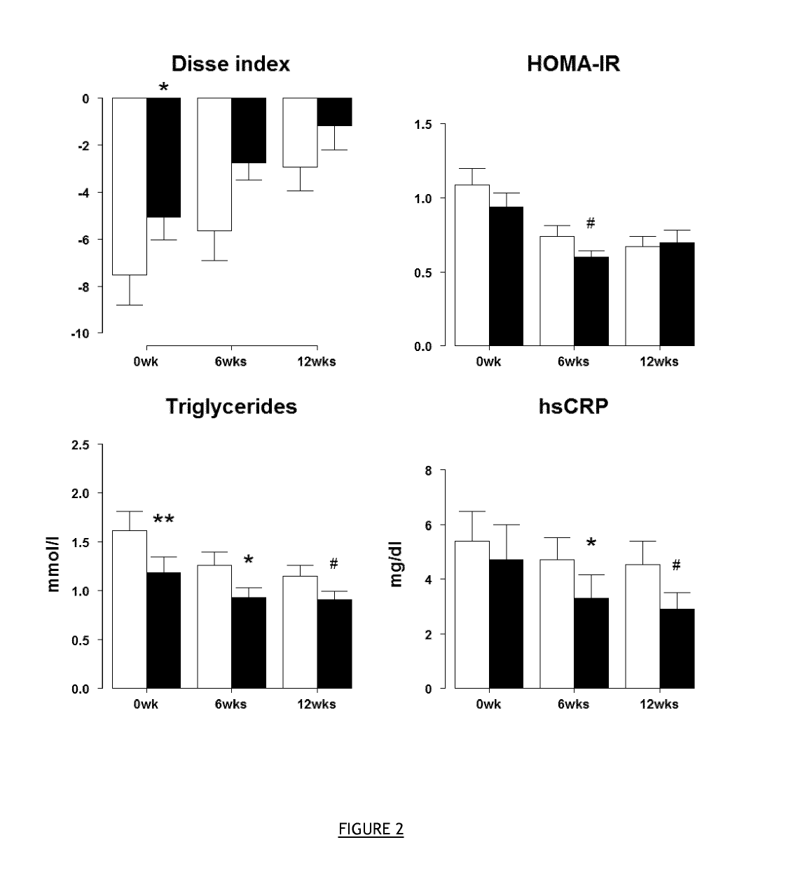

Two groups of patients with low gene count (LGC) and high gene count (HGC) were defined using the gene richness distribution (FIG. 1a). Genes significantly different in groups of individuals were identified by Mann-Whitney tests, at a p-value threshold of 0.0001. They were clustered by an abundance-based binning strategy, using the covariance of their gene frequency profiles among the individuals of the cohort. Spearman correlations coefficients were determined pairwise and all the genes that correlated above a rho coefficient of 0.85 were assigned to the same cluster. Abundance of a given species in each individual was estimated as a mean abundance of 25 arbitrarily selected ‘tracer’ genes for each cluster. The values were very close to the mean frequency of all the genes of a cluster.

### Receiver-Operator Characteristic (ROC) Analysis

The analyses were carried out to distinguish between HGC and LGC individuals by a combination of bacterial species. For each combination, only a single decision model was considered, computed as a sum of abundance of species more frequent in HGC than in LGC individuals. As opposed to the infinite number of regression models, such models are finite and can be exhaustively explored. To select the best models, the cross-validated area under the ROC curve (CV-AUC) criterion¹¹ was used, which is well adapted to classification models for binary outcome data.

### Correlations Between Microbial and Clinical Variables

Mann-Whitney tests were used to compare bioclinical variables, food items and gene clusters between LGC and HGC gene class at each time point. Associations between quantitative basal gene richness and bioclinical/food variables or deltas of bioclinical/food variables were investigated using linear models. For deltas, models were adjusted by the initial values of the variables. A p-value threshold of 0.05 was applied for statistical significance. No adjustment for multiple testing was used due to the highly correlated bioclinical and food variables. However, similar findings obtained in the accompanying paper¹⁰ support the results.

### Taxonomical Annotation

To obtain a precise and updated taxonomical annotation using BLASTN (BLAST 2.2.24, default parameters), the 3.3 million genes of the reference catalog were compared to the iMOMi database¹² (may 2011 release) containing 3,340 complete and draft microbial genomes downloaded from NCBI databanks. Following taxonomical assignation parameters described by Arumugam¹³, each gene was assigned with the taxonomy of the best-hit covering ≧80% of the gene length and according to the identity threshold for the taxonomic rank (≧65% for phylum, ≧85% for genus and ≧90% for species).

### Results

Forty-nine obese or overweight subjects were recruited and subjected to a 6-week energy restricted high-protein diet followed by a 6-week weight-maintenance diet the compliance was good, as indicated by a PCA analysis of 35 nutrients over time. Bioclinical characteristics and detailed qualitative and quantitative features of individuals' food intake were obtained at baseline, 6 and 12 weeks. The 35% decrease in energy intake after the first 6-weeks was associated with a reduction in body fat mass, adipocyte diameter and improvements of insulin sensitivity and markers of metabolism and inflammation. During the weight-maintenance phase, intake of nutrients tended to return to baseline values, while dietary total energy, carbohydrate and lipid intake remained lower than at beginning of the of the intervention Serum lipid variables tended to return to their basal levels as well, while a progressive reduction occurred in systemic inflammation markers.

The gut microbial composition of the study population was examined first at baseline. A bimodal distribution of bacterial gene number was observed (FIG. 1), similar to the one found in a cohort of 292 Danish individuals¹⁰, albeit somewhat less distinct, possibly due to a lower cohort size. At a threshold of 480,000 genes, defined visually and corresponding to that from the accompanying manuscript¹⁰, there were 18 (40%) low gene count (LGC) and 27 (60%) high gene count (HGC) individuals, harboring on average 379,436 and 561,499 genes, respectively, a one third difference. The enterotypes of the individuals in our cohort¹³ were determined. It was found that the Bacteroides- and Ruminococcus-driven enterotypes were prevalent among the LGC and HGC individuals, respectively, whereas the Prevotella-driven enterotype was distributed more uniformly (FIG. 1).

The baseline phenotypes of the study population were then examined. The LGC group had significantly higher insulin-resistance and fasting serum triglyceride levels, as well as a tendency towards higher inflammation than the HGC group (FIG. 2). It appears that in two European countries, the individuals of the LGC group, who have low gut bacterial richness, present phenotypes which expose them to an increased risk of obesity-associated co-morbidities. Antibiotic treatments, which lower the diversity, have been reported to improve the hormonal, metabolic and inflammatory status of obese mice; this apparent contradiction may be due to a restoration of a balance of the pro- and inflammatory bacterial species in mice. Interestingly, LGC subjects appeared to consume less fruits and vegetables and less fishery products than HGC subjects (Table 7), raising the possibility that the long-term dietary habits may affect gene richness and the associated phenotypes.

We next searched for the bacterial species differentially abundant in the LGC and the HGC groups. To this aim, the genes that had significantly different frequencies in the LGC and HGC groups were first identified. The genes from the same species were then clustered by a frequency-based covariance analysis, the genes that vary in a coordinated way are likely to belong to the same species¹⁰. 6,230 genes were identified that were different at Mann-Whitney p<0.0001; 4,462 (72%) were grouped into 112 clusters containing at least 2 genes at a Spearman correlation coefficient >0.85. A vast majority of these genes (3,966; 89%) were found in only 18 clusters, which are likely to originate from species differentially abundant in the LGC and HGC groups. Only two (Faecalibacterium prausnitzii and Roseburia inulinivorans) had a clear taxonomic assignment. The relative abundance of the 18 species in each individual was computed as a median frequency of their genes; all were significantly more abundant among the HGC individuals (FIG. 1).

To test whether the LGC and HGC individuals could be distinguished by the 18 species, an exhaustive ROC analysis of all species combinations was carried out, with 10-fold cross validation, using 90% of individuals for computation and the remaining 10% for test. The best AUC values for combination of different numbers of species are shown in FIG. 1; they ranged between 0.96 and 0.99 for 2 to 9 species combinations, indicating an almost perfect stratification of LGC and HGC individuals.

Interestingly, 14 of the 18 species (78%) were also identified as differentially abundant among the LGC and HGC individuals in a larger Danish cohort¹¹. Not surprisingly, the species combinations yielding the best AUC values for our cohort also efficiently stratified LGC and HGC Danes (FIG. 1). This indicates that the LGC and HGC individuals from two European countries differ in a similar way, not only by their clinical phenotypes but also by specific features of their gut microbiota.

Very interestingly, gene richness increased significantly in the LGC group after the energy-restricted diet and remained higher than at the baseline after the stabilization phase, even if a slight downwards trend was apparent, whereas it did not change significantly during intervention in the HGC group. Thus, a dietary intervention can correct a putative loss of richness in the LGC group, albeit partially, as the difference between the LGC and HGC groups remained significant at the end of the intervention. The intervention led to an improvement of metabolic variables in LGC individuals, bringing them closer to that of the HGC individuals: the tendency towards higher insulin resistance disappeared while the difference in triglyceride levels was attenuated (FIG. 2). Importantly, although the inflammation was decreased in all individuals, the difference between LGC and HGC individuals was not attenuated (FIG. 2).

To investigate the correlations between gene richness and dietary intervention further, those were studied in a quantitative way. For this purpose, we downsized the data set to 4.5 million uniquely mapping reads, enabling us to include at each time point 45 individuals. Prior to dietary intervention, gene richness was not related to age, sex, and BMI, but was associated with higher consumption of fruits and vegetables and lower triglyceride and cholesterol levels, confirming the LGC/HGC group comparisons. After 6 weeks of low-calorie diet, subjects with high gene richness at baseline had lower insulin-resistance and serum triglycerides as well as diminished adipose tissue inflammatory cells than the subjects with lower gene richness; the difference was significant for triglycerides and systemic inflammation after the weight maintenance phase, at 12 weeks. These observations indicate that gene richness affects the efficacy of the dietary intervention, even if the difference between the LGC and HGC individuals was globally attenuated. This conclusion was strengthened by the associations between higher gene richness at baseline and better improvement of adipose tissue and systemic inflammation (delta changes at 6 and 12 weeks, respectively). Gene richness appears to be predictive of the efficacy of dietary intervention in overweight/obese individuals.

TABLE 2
AUC obtained when determining low gut diversity by
detecting the presence or absence of 25 bacterial
genes from the given bacterial species.

| bacterial species | AUC |
|---|---|
| MO_HL_5 | 0.890946502057613 |
| MO_HL_6 | 0.890946502057613 |
| MO_HL_14 | 0.872427983539095 |
| MO_HL_9 | 0.845679012345679 |
| MO_HL_11 | 0.843621399176955 |
| MO_HL_13 | 0.831275720164609 |
| MO_HL_3 | 0.82716049382716 |
| MO_HL_8 | 0.826131687242798 |
| MO_HL_16 | 0.825102880658436 |
| MO_HL_4 | 0.820987654320988 |
| MO_HL_17 | 0.812757201646091 |
| MO_HL_1 | 0.808641975308642 |
| MO_HL_7 | 0.800411522633745 |
| MO_HL_12 | 0.79835390946502 |
| MO_HL_2 | 0.777777777777778 |
| MO_HL_10 | 0.765432098765432 |
| MO_HL_15 | 0.742798353909465 |
| MO_HL_18 | 0.740740740740741 |

TABLE 3
AUC above 0.9 are obtained when determining low gut diversity by detecting the
presence or absence of 25 bacterial genes from the given combination of 2 bacterial species.

| Combination of bacterial | | Combination of bacterial | |
|---|---|---|---|
| species | AUC | species | AUC |
| MO_HL_5 and MO_HL_6 | 0.948559670781893 | MO_HL_3 and MO_HL_13 | 0.874485596707819 |
| MO_HL_11 and MO_HL_12 | 0.948559670781893 | MO_HL_13 and MO_HL_14 | 0.874485596707819 |
| MO_HL_6 and MO_HL_7 | 0.94238683127572 | MO_HL_14 and MO_HL_15 | 0.874485596707819 |
| MO_HL_5 and MO_HL_11 | 0.938271604938272 | MO_HL_14 and MO_HL_17 | 0.874485596707819 |
| MO_HL_6 and MO_HL_8 | 0.932098765432099 | MO_HL_4 and MO_HL_8 | 0.872427983539095 |
| MO_HL_7 and MO_HL_11 | 0.932098765432099 | MO_HL_4 and MO_HL_14 | 0.872427983539095 |
| MO_HL_4 and MO_HL_13 | 0.92798353909465 | MO_HL_9 and MO_HL_15 | 0.872427983539094 |
| MO_HL_5 and MO_HL_12 | 0.921810699588477 | MO_HL_1 and MO_HL_3 | 0.87037037037037 |
| MO_HL_6 and MO_HL_13 | 0.921810699588477 | MO_HL_1 and MO_HL_5 | 0.87037037037037 |
| MO_HL_13 and MO_HL_17 | 0.919753086419753 | MO_HL_1 and MO_HL_13 | 0.868312757201646 |
| MO_HL_5 and MO_HL_17 | 0.91358024691358 | MO_HL_3 and MO_HL_16 | 0.868312757201646 |
| MO_HL_6 and MO_HL_12 | 0.91358024691358 | MO_HL_11 and MO_HL_15 | 0.868312757201646 |
| MO_HL_5 and MO_HL_7 | 0.911522633744856 | MO_HL_13 and MO_HL_15 | 0.864197530864198 |
| MO_HL_5 and MO_HL_8 | 0.909465020576132 | MO_HL_3 and MO_HL_12 | 0.864197530864197 |
| MO_HL_8 and MO_HL_11 | 0.909465020576132 | MO_HL_5 and MO_HL_9 | 0.864197530864197 |
| MO_HL_3 and MO_HL_9 | 0.907407407407407 | MO_HL_6 and MO_HL_9 | 0.864197530864197 |
| MO_HL_5 and MO_HL_13 | 0.907407407407407 | MO_HL_7 and MO_HL_18 | 0.864197530864197 |
| MO_HL_14 and MO_HL_16 | 0.905349794238683 | MO_HL_2 and MO_HL_15 | 0.863168724279835 |
| MO_HL_12 and MO_HL_13 | 0.904320987654321 | MO_HL_1 and MO_HL_11 | 0.862139917695473 |
| MO_HL_2 and MO_HL_7 | 0.901234567901235 | MO_HL_3 and MO_HL_6 | 0.862139917695473 |
| MO_HL_2 and MO_HL_14 | 0.89917695473251 | MO_HL_8 and MO_HL_17 | 0.862139917695473 |
| MO_HL_4 and MO_HL_5 | 0.89917695473251 | MO_HL_13 and MO_HL_18 | 0.862139917695473 |
| MO_HL_11 and MO_HL_17 | 0.89917695473251 | MO_HL_7 and MO_HL_9 | 0.860082304526749 |
| MO_HL_4 and MO_HL_11 | 0.898148148148148 | MO_HL_8 and MO_HL_18 | 0.860082304526749 |
| MO_HL_7 and MO_HL_12 | 0.896090534979424 | MO_HL_9 and MO_HL_16 | 0.860082304526749 |
| MO_HL_1 and MO_HL_7 | 0.895061728395062 | MO_HL_1 and MO_HL_17 | 0.858024691358025 |
| MO_HL_1 and MO_HL_8 | 0.895061728395062 | MO_HL_5 and MO_HL_15 | 0.858024691358025 |
| MO_HL_8 and MO_HL_12 | 0.895061728395062 | MO_HL_3 and MO_HL_10 | 0.858024691358024 |
| MO_HL_2 and MO_HL_5 | 0.893004115226337 | MO_HL_1 and MO_HL_15 | 0.8559670781893 |
| MO_HL_2 and MO_HL_13 | 0.893004115226337 | MO_HL_9 and MO_HL_10 | 0.8559670781893 |
| MO_HL_3 and MO_HL_14 | 0.893004115226337 | MO_HL_8 and MO_HL_13 | 0.854938271604938 |
| MO_HL_6 and MO_HL_11 | 0.890946502057613 | MO_HL_2 and MO_HL_16 | 0.853909465020576 |
| MO_HL_9 and MO_HL_14 | 0.890946502057613 | MO_HL_8 and MO_HL_9 | 0.853909465020576 |
| MO_HL_6 and MO_HL_17 | 0.888888888888889 | MO_HL_4 and MO_HL_18 | 0.851851851851852 |
| MO_HL_2 and MO_HL_4 | 0.887860082304527 | MO_HL_9 and MO_HL_12 | 0.851851851851852 |
| MO_HL_4 and MO_HL_6 | 0.886831275720165 | MO_HL_9 and MO_HL_17 | 0.851851851851852 |
| MO_HL_7 and MO_HL_13 | 0.885802469135803 | MO_HL_10 and MO_HL_13 | 0.849794238683127 |
| MO_HL_4 and MO_HL_7 | 0.885802469135802 | MO_HL_11 and MO_HL_18 | 0.849794238683127 |
| MO_HL_2 and MO_HL_6 | 0.88477366255144 | MO_HL_1 and MO_HL_2 | 0.847736625514403 |
| MO_HL_2 and MO_HL_8 | 0.88477366255144 | MO_HL_3 and MO_HL_17 | 0.847736625514403 |
| MO_HL_2 and MO_HL_9 | 0.88477366255144 | MO_HL_1 and MO_HL_6 | 0.845679012345679 |
| MO_HL_3 and MO_HL_7 | 0.88477366255144 | MO_HL_3 and MO_HL_18 | 0.845679012345679 |
| MO_HL_3 and MO_HL_8 | 0.88477366255144 | MO_HL_4 and MO_HL_9 | 0.845679012345679 |
| MO_HL_6 and MO_HL_15 | 0.88477366255144 | MO_HL_8 and MO_HL_15 | 0.845679012345679 |
| MO_HL_9 and MO_HL_13 | 0.88477366255144 | MO_HL_1 and MO_HL_16 | 0.843621399176955 |
| MO_HL_1 and MO_HL_9 | 0.882716049382716 | MO_HL_3 and MO_HL_11 | 0.843621399176954 |
| MO_HL_2 and MO_HL_3 | 0.882716049382716 | MO_HL_12 and MO_HL_17 | 0.842592592592593 |
| MO_HL_2 and MO_HL_11 | 0.882716049382716 | MO_HL_2 and MO_HL_12 | 0.842592592592592 |
| MO_HL_10 and MO_HL_14 | 0.882716049382716 | MO_HL_2 and MO_HL_17 | 0.84156378600823 |
| MO_HL_11 and MO_HL_13 | 0.882716049382716 | MO_HL_7 and MO_HL_10 | 0.84156378600823 |
| MO_HL_3 and MO_HL_4 | 0.880658436213992 | MO_HL_1 and MO_HL_12 | 0.839506172839506 |
| MO_HL_5 and MO_HL_18 | 0.880658436213992 | MO_HL_3 and MO_HL_5 | 0.839506172839506 |
| MO_HL_7 and MO_HL_8 | 0.880658436213992 | MO_HL_3 and MO_HL_15 | 0.837448559670782 |
| MO_HL_7 and MO_HL_17 | 0.880658436213992 | MO_HL_7 and MO_HL_15 | 0.83641975308642 |
| MO_HL_9 and MO_HL_11 | 0.878600823045268 | MO_HL_6 and MO_HL_18 | 0.835390946502058 |
| MO_HL_6 and MO_HL_14 | 0.878600823045267 | MO_HL_7 and MO_HL_16 | 0.835390946502058 |
| MO_HL_1 and MO_HL_14 | 0.876543209876543 | MO_HL_8 and MO_HL_16 | 0.835390946502058 |
| MO_HL_5 and MO_HL_14 | 0.876543209876543 | MO_HL_13 and MO_HL_16 | 0.835390946502058 |
| MO_HL_7 and MO_HL_14 | 0.876543209876543 | MO_HL_4 and MO_HL_16 | 0.835390946502057 |
| MO_HL_8 and MO_HL_14 | 0.876543209876543 | MO_HL_8 and MO_HL_10 | 0.835390946502057 |
| MO_HL_9 and MO_HL_18 | 0.876543209876543 | MO_HL_6 and MO_HL_10 | 0.833333333333333 |
| MO_HL_11 and MO_HL_14 | 0.876543209876543 | MO_HL_6 and MO_HL_16 | 0.833333333333333 |
| MO_HL_12 and MO_HL_14 | 0.876543209876543 | MO_HL_10 and MO_HL_16 | 0.833333333333333 |
| MO_HL_14 and MO_HL_18 | 0.876543209876543 | MO_HL_11 and MO_HL_16 | 0.833333333333333 |
| MO_HL_2 and MO_HL_10 | 0.874485596707819 | MO_HL_1 and MO_HL_4 | 0.831275720164609 |
| | | MO_HL_15 and MO_HL_17 | 0.831275720164609 |

TABLE 4
AUC above 0.95 are obtained when determining low gut diversity
by detecting the presence or absence of 25 bacterial genes from
the given combination of 3 bacterial species.

| Combination of bacterial species | AUC |
|---|---|
| MO_HL_7, MO_HL_11 and MO_HL_12 | 0.993827160493827 |
| MO_HL_5, MO_HL_11 and MO_HL_12 | 0.977366255144033 |
| MO_HL_8, MO_HL_11 and MO_HL_12 | 0.967078189300411 |
| MO_HL_4, MO_HL_7 and MO_HL_11 | 0.965020576131687 |
| MO_HL_5, MO_HL_7 and MO_HL_11 | 0.965020576131687 |
| MO_HL_7, MO_HL_11 and MO_HL_17 | 0.958847736625514 |
| MO_HL_5, MO_HL_6 and MO_HL_8 | 0.954732510288066 |
| MO_HL_7, MO_HL_12 and MO_HL_13 | 0.954732510288066 |
| MO_HL_5, MO_HL_6 and MO_HL_12 | 0.952674897119342 |
| MO_HL_6, MO_HL_7 and MO_HL_12 | 0.952674897119342 |
| MO_HL_7, MO_HL_8 and MO_HL_11 | 0.952674897119342 |
| MO_HL_4, MO_HL_5 and MO_HL_11 | 0.950617283950617 |
| MO_HL_6, MO_HL_8 and MO_HL_11 | 0.950617283950617 |

TABLE 5
AUC above 0.96 are obtained when determining low gut diversity by detecting the
presence or absence of 25 bacterial genes from the given combination of 4 bacterial species

| Combination of bacterial species | AUC |
|---|---|
| MO_HL_5, MO_HL_7, MO_HL_11 and MO_HL_12 | 0.987654320987654 |
| MO_HL_7, MO_HL_8, MO_HL_11 and MO_HL_12 | 0.981481481481482 |
| MO_HL_7, MO_HL_11, MO_HL_12 and MO_HL_17 | 0.981481481481481 |
| MO_HL_6, MO_HL_8, MO_HL_11 and MO_HL_12 | 0.977366255144033 |
| MO_HL_2, MO_HL_7, MO_HL_11 and MO_HL_12 | 0.975308641975309 |
| MO_HL_7, MO_HL_11, MO_HL_12 and MO_HL_15 | 0.975308641975309 |
| MO_HL_2, MO_HL_8, MO_HL_11 and MO_HL_2 | 0.973251028806584 |
| MO_HL_5, MO_HL_11, MO_HL_12 and MO_HL_17 | 0.973251028806584 |
| MO_HL_6, MO_HL_7, MO_HL_11 and MO_HL_12 | 0.973251028806584 |
| MO_HL_5, MO_HL_8, MO_HL_11 and MO_HL_12 | 0.97119341563786 |
| MO_HL_8, MO_HL_11, MO_HL_12 and MO_HL_17 | 0.97119341563786 |
| MO_HL_7, MO_HL_11, MO_HL_12 and MO_HL_13 | 0.969135802469136 |
| MO_HL_5, MO_HL_6, MO_HL_8 and MO_HL_11 | 0.967078189300411 |
| MO_HL_7, MO_HL_8, MO_HL_11 and MO_HL_17 | 0.967078189300411 |
| MO_HL_2, MO_HL_5, MO_HL_11 and MO_HL_12 | 0.965020576131687 |
| MO_HL_4, MO_HL_7, MO_HL_11 and MO_HL_12 | 0.965020576131687 |
| MO_HL_5, MO_HL_7, MO_HL_8 and MO_HL_11 | 0.965020576131687 |
| MO_HL_7, MO_HL_8, MO_HL_12 and MO_HL_13 | 0.965020576131687 |
| MO_HL_7, MO_HL_12, MO_HL_13 and MO_HL_17 | 0.965020576131687 |
| MO_HL_2, MO_HL_4, MO_HL_7 and MO_HL_11 | 0.962962962962963 |
| MO_HL_2, MO_HL_7, MO_HL_8 and MO_HL_13 | 0.962962962962963 |
| MO_HL_2, MO_HL_7, MO_HL_12 and MO_HL_13 | 0.962962962962963 |
| MO_HL_4, MO_HL_5, MO_HL_7 and MO_HL_11 | 0.962962962962963 |
| MO_HL_5, MO_HL_6, MO_HL_12 and MO_HL_13 | 0.962962962962963 |
| MO_HL_5, MO_HL_11, MO_HL_12 and MO_HL_13 | 0.962962962962963 |
| MO_HL_6, MO_HL_8, MO_HL_12 and MO_HL_13 | 0.962962962962963 |
| MO_HL_2, MO_HL_7, MO_HL_8 and MO_HL_11 | 0.960905349794239 |
| MO_HL_5, MO_HL_6, MO_HL_7 and MO_HL_8 | 0.960905349794239 |
| MO_HL_5, MO_HL_6, MO_HL_11 and MO_HL_12 | 0.960905349794239 |
| MO_HL_6, MO_HL_7, MO_HL_8 and MO_HL_11 | 0.960905349794239 |
| MO_HL_6, MO_HL_7, MO_HL_8 and MO_HL_12 | 0.960905349794239 |
| MO_HL_6, MO_HL_7, MO_HL_12 and MO_HL_13 | 0.960905349794239 |
| MO_HL_7, MO_HL_8, MO_HL_11 and MO_HL_15 | 0.960905349794239 |
| MO_HL_11, MO_HL_12, MO_HL_13 and MO_HL_17 | 0.960905349794239 |

TABLE 6
bacterial species and combinations of 2 to 18 bacterial species giving the best AUC
in the method of the invention.

| Bacterial species and combination thereof | AUC |
|---|---|
| MO_HL_5 | 0.890946502 |
| MO_HL_12 and MO_HL_11 | 0.948559671 |
| MO_HL_11, MO_HL_7 and MO_HL_12 | 0.99382716 |
| MO_HL_11, MO_HL_5, MO_HL_7 and MO_HL_12 | 0.987654321 |
| MO_HL_11, MO_HL_8, MO_HL_12, MO_HL_7 and MO_HL_2 | 0.983539095 |
| MO_HL_17, MO_HL_5, MO_HL_12, MO_HL_7, MO_HL_11and MO_HL_8 | 0.981481481 |
| MO_HL_12, MO_HL_11, MO_HL_2, MO_HL_5, MO_HL_8, MO_HL_13 | 0.983539095 |
| and MO_HL_7 | |
| MO_HL_12, MO_HL_2, MO_HL_17, MO_HL_11, MO_HL_13, MO_HL_5, | 0.983539095 |
| MO_HL_8 and MO_HL_7 | |
| MO_HL_4, MO_HL_11, MO_HL_6, MO_HL_13, MO_HL_2, MO_HL_8, | 0.979423868 |
| MO_HL_12, MO_HL_7 and MO_HL_17 | |
| MO_HL_8, MO_HL_11, MO_HL_6, MO_HL_4, MO_HL_13, MO_HL_5, | 0.981481481 |
| MO_HL_2, MO_HL_7, MO_HL_17 and MO_HL_12 | |
| MO_HL_5, MO_HL_13, MO_HL_4, MO_HL_12, MO_HL_15, MO_HL_17, | 0.977366255 |
| MO_HL_6, MO_HL_2, MO_HL_8, MO_HL_7 and MO_HL_11 | |
| MO_HL_11, MO_HL_6, MO_HL_17, MO_HL_4, MO_HL_3, MO_HL_12, | 0.975308642 |
| MO_HL_5, MO_HL_10, MO_HL_8, MO_HL_7, MO_HL_2 and MO_HL_13 | |
| MO_HL_18, MO_HL_15, MO_HL_11, MO_HL_10, MO_HL_5, MO_HL_4, | 0.973251029 |
| MO_HL_6, MO_HL_8, MO_HL_12, MO_HL_7, MO_HL_2, MO_HL_13 and | |
| MO_HL_3 | |
| MO_HL_6, MO_HL_17, MO_HL_3, MO_HL_2, MO_HL_4, MO_HL_18, | 0.971193416 |
| MO_HL_5, MO_HL_13, MO_HL_10, MO_HL_15, MO_HL_7, MO_HL_1, | |
| MO_HL_8 and MO_HL_12 | |
| MO_HL_10, MO_HL_18, MO_HL_2, MO_HL_13, MO_HL_7, MO_HL_1, | 0.971193416 |
| MO_HL_11, MO_HL_3, MO_HL_4, MO_HL_15, MO_HL_5, MO_HL_6, | |
| MO_HL_17, MO_HL_12 and MO_HL_8 | |
| MO_HL_7, MO_HL_4, MO_HL_13, MO_HL_1, MO_HL_5, MO_HL_6, | 0.946502058 |
| MO_HL_11, MO_HL_12, MO_HL_8, MO_HL_3, MO_HL_18, MO_HL_2, | |
| MO_HL_10, MO_HL_17, MO_HL_15 and MO_HL_9 | |
| MO_HL_10, MO_HL_8, MO_HL_1, MO_HL_16, MO_HL_18, MO_HL_5, | 0.942386831 |
| MO_HL_15, MO_HL_2, MO_HL_7, MO_HL_6, MO_HL_13, MO_HL_11, | |
| MO_HL_17, MO_HL_3, MO_HL_14, MO_HL_4 and MO_HL_12 | |
| MO_HL_15, MO_HL_17, MO_HL_4, MO_HL_14, MO_HL_16, MO_HL_10, | 0.940329218 |
| MO_HL_7, MO_HL_6, MO_HL_11, MO_HL_9, MO_HL_5, MO_HL_3, | |
| MO_HL_8, MO_HL_1, MO_HL_12, MO_HL_18, MO_HL_13 and MO_HL_2 | |

### REFERENCES

1. Rizkalla, S. W. et al. Differential effects of macronutrient content in 2 energy-restricted diets on cardiovascular risk factors and adipose tissue cell size in moderately obese individuals: a randomized controlled trial. Am. J. Clin. Nutr. 95, 49-63 (2012).
2. Bouché, C. et al. Five-week, low-glycemic index diet decreases total fat mass and improves plasma lipid profile in moderately overweight nondiabetic men. Diabetes Care 25, 822-828 (2002).
3. Tordjman, J. et al. Structural and inflammatory heterogeneity in subcutaneous adipose tissue: Relation with liver histopathology in morbid obesity. Journal of Hepatology (2012).doi:10.1016/j.jhep.2011.12.015
4. Disse, E. et al. A lipid-parameter-based index for estimating insulin sensitivity and identifying insulin resistance in a healthy population. Diabetes Metab. 34, 457-463 (2008).
5. Antuna-Puente, B. et al. Evaluation of insulin sensitivity with a new lipid-based index in non-diabetic postmenopausal overweight and obese women before and after a weight loss intervention. Eur. J. Endocrinol. 161, 51-56 (2009).
6. Prat-Larquemin, L. et al. Adipose angiotensinogen secretion, blood pressure, and AGT M235T polymorphism in obese patients. Obes. Res. 12, 556-561 (2004).
7. Benjamini, Y. & Hochberg, Y. Controlling the false discovery rate: a practical and powerful approach to multiple testing. Journal of the Royal Statistical Society. Series B. Methodological 57, 289-300
8. Qin, J. et al. A human gut microbial gene catalogue established by metagenomic sequencing. Nature 464, 59-65 (2010).
9. Pons, N. et al. METEOR, a platform for quantitative metagenomic profiling of complex ecosystems. JOBIM (2010).
10. Le Chatelier, E. et al. Richness of human gut microbial communities correlates with metabolic markers. submitted in Nature
11. Jiang, D., Huang, J. & Zhang, Y. The Cross-Validated AUC for MCP-Logistic Regression with High-Dimensional Data. Stat Methods Med Res (2011).doi:10.1177/0962280211428385
12. Pons, N., Batto, J.-M., Ehrlich, S. D. & Renault, P. Development of software facilities to characterize regulatory binding motifs and application to streptococcaceae. J. Mol. Microbiol. Biotechnol. 14, 67-73 (2008).
13. Arumugam, M. et al. Enterotypes of the human gut microbiome. Nature 473, 174-180 (2011).

1. A method for determining whether an overweight subject has a reduced gut bacterial diversity, said method comprising:
a) detecting from a gut microbial DNA sample obtained from said subject whether at least one gene from at least one bacterial species from Table 1 is absent in said sample, and
b) determining that the subject has a reduced gut bacterial diversity, if at least one gene from at least one bacterial species from Table 1 is absent in said sample.

2. A method according to claim 1, characterized in that it comprises a step of detecting from a gut microbial DNA sample obtained from said subject whether at least one gene from a bacterial species chosen from the list consisting in MO_HL_{—}5, MO_HL_{—}6, MO_HL_{—}14, MO_HL_{—}9, MO_HL_{—}11, MO_HL_{—}13, MO_HL_{—}3, MO_HL_{—}8, MO_HL_{—}16, MO_HL_{—}4, MO_HL_{—}17, MO_HL_{—}1, or MO_HL_{—}7 from Table 1 is absent in said sample.

3. A method according to claim 1 or 2, characterized in that comprises a step of detecting from a gut microbial DNA sample obtained from said subject whether at least one gene from the bacterial species MOHL-5 from Table 1 is absent in said sample.

4. Method according to any of claims 1 to 3, characterized in that it comprises a step of detecting from a gut microbial DNA sample obtained from said subject whether at least one gene from each of the bacterial species of any of the bacterial species combinations indicated in Table 3, 4 and/or 5 is absent in said sample.

5. Method according to any of claims 1 to 3, characterized in that it comprises a step of detecting from a gut microbial DNA sample obtained from said subject whether:
at least one gene from each of the bacterial species MO_HL_{—}12 and MO_HL_{—}11 from Table 1 are absent in said sample, or;
at least one gene from each of the bacterial species MO_HL_{—}11, MO_HL_{—}7 and MO_HL_{—}12 from Table 1 are absent in said sample, or;
at least one gene from each of the bacterial species MO_HL_{—}11, MO_HL_{—}5, MO_HL_{—}7 and MO_HL_{—}12 from Table 1 are absent in said sample, or;
at least one gene from each of the bacterial species MO_HL_{—}11, MO_HL_{—}8, MO_HL_{—}12, MO_HL_{—}7 and MO_HL_{—}2 from Table 1 are absent in said sample, or;
at least one gene from each of the bacterial species MO_HL_{—}17, MO_HL_{—}5, MO_HL_{—}12, MO_HL_{—}7, MO_HL_{—}11 and MO_HL_{—}8 from Table 1 are absent in said sample, or;
at least one gene from each of the bacterial species MO_HL_{—}12, MO_HL_{—}11, MO_HL_{—}2, MO_HL_{—}5, MO_HL_{—}8, MO_HL_{—}13 and MO_HL_{—}7 from Table 1 are absent in said sample, or;
at least one gene from each of the bacterial species MO_HL_{—}12, MO_HL_{—}2, MO_HL_{—}17, MO_HL_{—}11, MO_HL_{—}13, MO_HL_{—}5, MO_HL_{—}8 and MO_HL_{—}7 from Table 1 are absent in said sample, or;
at least one gene from each of the bacterial species MO_HL_{—}4, MO_HL_{—}11, MO_HL_{—}6, MO_HL_{—}13, MO_HL_{—}2, MO_HL_{—}8, MO_HL_{—}12, MO_HL_{—}7 and MO_HL_{—}17 from Table 1 are absent in said sample, or;
at least one gene from each of the bacterial species MO_HL_{—}8, MO_HL_{—}11, MO_HL_{—}6, MO_HL_{—}4, MO_HL_{—}13, MO_HL_{—}5, MO_HL_{—}2, MO_HL_{—}7, MO_HL_{—}17 and MO_HL_{—}12 from Table 1 are absent in said sample;
at least one gene from each of the bacterial species MO_HL_{—}5, MO_HL_{—}13, MO_HL_{—}4, MO_HL_{—}12, MO_HL_{—}15, MO_HL_{—}17, MO_HL_{—}6, MO_HL_{—}2, MO_HL_{—}8, MO_HL_{—}7 and MO_HL_{—}11 from Table 1 are absent in said sample;
at least one gene from each of the bacterial species MO_HL_{—}11, MO_HL_{—}6, MO_HL_{—}17, MO_HL_{—}4, MO_HL_{—}3, MO_HL_{—}12, MO_HL_{—}5, MO_HL_{—}10, MO_HL_{—}8, MO_HL_{—}7, MO_HL_{—}2 and MO_HL_{—}13 from Table 1 are absent, in said sample;
at least one gene from each of the bacterial species MO_HL_{—}18, MO_HL_{—}15, MO_HL_{—}11, MO_HL_{—}10, MO_HL_{—}5, MO_HL_{—}4, MO_HL_{—}6, MO_HL_{—}8, MO_HL_{—}12, MO_HL_{—}7, MO_HL_{—}2, MO_HL_{—}13 and MO_HL_{—}3 from Table 1 are absent in said sample, or;
at least one gene from each of the bacterial species MO_HL_{—}6, MO_HL_{—}17, MO_HL_{—}3, MO_HL_{—}2, MO_HL_{—}4, MO_HL_{—}18, MO_HL_{—}5, MO_HL_{—}13, MO_HL_{—}10, MO_HL_{—}15, MO_HL_{—}7, MO_HL_{—}1, MO_HL_{—}8 and MO_HL_{—}12 from Table 1 are absent in said sample, or;
at least one gene from each of the bacterial species MO_HL_{—}10, MO_HL_{—}18, MO_HL_{—}2, MO_HL_{—}13, MO_HL_{—}7, MO_HL_{—}1, MO_HL_{—}11, MO_HL_{—}3, MO_HL_{—}4, MO_HL_{—}15, MO_HL_{—}5, MO_HL_{—}6, MO_HL_{—}17, MO_HL_{—}12 and MO_HL_{—}8 from Table 1 are absent, in said sample;
at least one gene from each of the bacterial species MO_HL_{—}7, MO_HL_{—}4, MO_HL_{—}13, MO_HL_{—}1, MO_HL_{—}5, MO_HL_{—}6, MO_HL_{—}11, MO_HL_{—}12, MO_HL_{—}8, MO_HL_{—}3, MO_HL_{—}18, MO_HL_{—}2, MO_HL_{—}10, MO_HL_{—}17, MO_HL_{—}15 and MO_HL_{—}9 from Table 1 are absent in said sample, or;
at least one gene from each of the bacterial species MO_HL_{—}10, MO_HL_{—}8, MO_HL_{—}1, MO_HL_{—}16, MO_HL_{—}18, MO_HL_{—}5, MO_HL_{—}15, MO_HL_{—}2, MO_HL_{—}7, MO_HL_{—}6, MO_HL_{—}13, MO_HL_{—}11, MO_HL_{—}17, MO_HL_{—}3, MO_HL_{—}14, MO_HL_{—}4 and MO_HL_{—}12 from Table 1 are absent, in said sample;
at least one gene from each of the bacterial species MO_HL_{—}15, MO_HL_{—}17, MO_HL_{—}4, MO_HL_{—}14, MO_HL_{—}16, MO_HL_{—}10, MO_HL_{—}7, MO_HL_{—}6, MO_HL_{—}11, MO_HL_{—}9, MO_HL_{—}5, MO_HL_{—}3, MO_HL_{—}8, MO_HL_{—}1, MO_HL_{—}12, MO_HL_{—}18, MO_HL_{—}13 and MO_HL_{—}2 from Table 1 are absent, in said sample.

6. A method according to any of claims 1 to 5, characterized in that it comprises detecting the number of copies of at least one bacterial gene from said bacterial species in the sample.

7. A method according to claim 6, characterized in that it comprises detecting the number of copies of at least 10, 15, 20 or at least 25 bacterial genes from said bacterial species in the sample.

8. A method according to any of claim 6 or 7, characterized in that said bacterial genes are chosen in the list consisting of sequence SEQID 1 to sequence SEQ ID 450.

9. A method according to any of claims 1 to 8, characterized in that the presence or absence of the bacterial genes according to the invention is detected by the use of a nucleic microarray.

10. A method according to claim 9, characterized in that the nucleic microarray is an oligonucleotide microarray comprising at least one oligonucleotide specific for at least one gene having a sequence selected from SEQ ID NOs 1-450.

11. Method for determining if an overweight subject is at risk to develop obesity related co-morbidities, said method comprising the steps of:
c) determining that said subject has a reduced gut bacterial diversity with a method according to anyone of claims 1 to 10;
d) if said subject has a reduced gut bacterial diversity, determining that said overweight subject is at is at risk to develop obesity related co-morbidities.

12. A method for determining if an overweight subject is in need of a diet, said method comprising the steps of:
a) determining that said subject has a reduced gut bacterial diversity with a method according to anyone of claims 1 to 10;
b) if said subject has a reduced gut bacterial diversity, determining that said subject is in need of a diet.

13. A method for alleviating the risks to develop obesity-related co-morbidities, said method comprising the steps of:
a) determining that said subject has a reduced gut bacterial diversity with a method according to anyone of claims 1 to 10;
b) if said subject has a reduced gut bacterial diversity, determining that said subject is in need of a diet.

14. A method for determining if an overweight subject is in need of a low-grade inflammation treatment, preferably in addition to the implementation of a diet, comprising the steps of:
a) determining that said subject has a reduced gut bacterial diversity with a method according to anyone of claims 1 to 10;
b) if said subject has a reduced gut bacterial diversity, determining that said subject is in need of a low-grade inflammation treatment, preferably in addition to the implementation of a diet.

15. A method for monitoring the efficiency of a diet in increasing gut bacterial microbiome richness in an overweight subject in need thereof, said method comprising the steps of:
a) determining that said subject has a reduced gut bacterial diversity with a method according to anyone of claims 1 to 10;
b) implementing said diet;
c) determining from a second gut microbial DNA sample obtained from said subject if said subject has a reduced gut bacterial diversity with a method according to anyone of claims 1 to 10;
d) if said subject has a reduced gut bacterial diversity, determining that the diet is efficient in increasing gut bacterial microbiome richness in said subject.
